(19) United States
(12) Patent Application Publication
(10) Pub. No.: US 2006/0224044 A1
(43) Pub. Date: Oct. 5, 2006

(54) Surgical retractors and methods of use

(75) Inventors: Connie Marchek, Foxboro, MA (US); Michael Michielli, Medway, MA (US); Jonathan Haylock, Playa Vista, CA (US); Thomas W. Higginbotham, Independence, MO (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuy Spine, Inc., Raynham, MA (US)

(21) Appl. No.: 11/095,757

(22) Filed: Mar. 31, 2005

(51) Int. Cl. A61B 1/32

(52) U.S. Cl. 600/233

(57) ABSTRACT

Described herein are surgical retractors that include a frame and a blade assembly. The blade assembly includes an arm extending from the frame, a blade attached to the arm and moveable relative to the frame, and a locking mechanism. The locking mechanism is moveable from a first position to a second position to prevent movement of the blade in at least two, non-collinear directions. Also described are methods of performing a surgical procedure using the surgical retractors.

### BACKGROUND OF THE INVENTION

In surgical procedures, it is important to minimize trauma to the patient and damage to the tissue as much as possible. For that reason, surgeons try to keep incisions to a minimum while performing surgical procedures. However, the surgeon performing a delicate surgery must still be able to have a clear view of the operating field.

A wide variety of retractors are available to keep an incision open and provide a clear view of the operating field. Generally, surgical retractors are used in surgical operations to reposition muscular tissue, vessels, nerves, and other tissue with the aid of retractor blades, thereby providing access to the site of the operation. Surgeons utilize retractors in an attempt to achieve the goal of keeping the incision to a minimum while still providing a clear view of the operating field. Surgical retractors are particularly important in performing surgical procedures that involve the spinal column, where access to the surgical sight can be obtained through a posterior, posterior-lateral, anterior, lateral, or an anterior-lateral approach.

A need exists for new surgical retractors that provide the surgeon with a greater flexibility to explore and create an operating field and perform surgical procedures in a minimally invasive manner.

### SUMMARY OF THE INVENTION

In some embodiments, this invention includes surgical retractors. In one embodiment, the surgical retractor comprises a frame and a blade assembly. The blade assembly includes an arm extending from the frame, a blade attached to the arm and moveable relative to the frame, and a locking mechanism. The locking mechanism is moveable from a first position to a second position to prevent movement of the blade in at least two, non-collinear directions. In further embodiments, the locking mechanism is moveable from a first position to a second position to prevent movement of the blade in at least three, non-collinear directions.

In other embodiments, this invention includes methods of forming a surgical site in a mammal. In one embodiment, the method comprises creating an incision in the mammal, directing a retractor through the incision and into the mammal, and retracting the tissue of the mammal at the incision with the retractor to form a surgical site. The retractor includes a frame and a blade assembly. The blade assembly includes an arm extending from the frame, and a blade attached to the arm and moveable relative to the frame, and a locking mechanism that is moveable from a first position to a second position to prevent movement of the blade in at least two, non-collinear directions. The surgical site is defined at least in part by the blades.

This invention provides simple, easy to use, low-profile frame-based surgical retractors that can be used to perform minimally invasive surgery on a mammal (e.g., a human). In some embodiments, the surgical retractors of the invention are assembled to an obtruator and inserted into a mammal through a minimal insertion, thereby eliminating the need for stepwise dilation of the incision. Once inserted into a mammal, the blades of the surgical retractors can be individually retracted, rotated or “toed in,” and/or extended, thereby allowing a practitioner of the invention to create and customize a working space for surgical procedures while reducing the length of an incision and minimizing muscle creep during a procedure. The surgical retractors of this invention also provide space to attach additional blades, light sources, nerve root retractors, and/or other surgical instrumentation.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 illustrates a perspective view of an embodiment of the invention.

FIGS. 2A and 2B illustrate perspective views of a portion of a retractor of the invention.

FIG. 2C illustrate an exploded view of a portion of a retractor of the invention.

FIGS. 3A and 3B include two perspective views from different angles of a bottom or ventral portion of a retractor of the invention.

FIGS. 4A and 4B illustrates various views of a portion of a retractor of the invention that includes a blade.

FIG. 5 illustrates an embodiment of the invention that includes a portion of a retractor.

FIG. 6A-6G illustrate various views of a retractor of the invention.

FIG. 7 illustrates a view of the distal end of the outer face of a blade of a retractor of the invention.

FIG. 8 illustrates a side view of a portion of a blade and a portion of an arm of a retractor of the invention.

FIG. 9A-9H illustrate various views of a retractor system of the invention and portions of a retractor system of the invention.

FIG. 10A-10D illustrate various views of portions of a retractor of the invention.

FIG. 11A-11D illustrate various views of retractors of the invention and portions of retractors of the invention.

FIG. 12A-12C illustrate views of a retractor of the invention at various degrees of retraction.

FIG. 13A-13E illustrate various views of a retractor of the invention and portions of a retractor of the invention.

FIGS. 14A-14H illustrate one embodiment of a method of the invention where a retractor of the invention is directed into a mammal to the depth of the surgical site or near the depth of the surgical site.

### DETAILED DESCRIPTION OF THE INVENTION

A description of preferred embodiments of the invention follows. While this invention has been particularly shown and described with references to preferred embodiments thereof, it will be understood by those skilled in the art that various changes in form and details may be made therein without departing from the scope of the invention encompassed by the appended claims.

As used herein, the articles “a” and “an” refer to one or to more than one (i.e. to at least one) of the grammatical object of the article. For example, “an element” means one element or more than one element.

The terms “comprise,” “include,” “have,” and derivatives thereof are used herein interchangeably as comprehensive, open-ended terms. For example, use of “comprising,” “including,” or “having” means that whatever element is comprised, had, or included, is not the only element encompassed by the subject of the clause that contains the verb.

In some embodiments, the invention includes a surgical retractor comprising a frame and a blade assembly. The blade assembly includes an arm extending from the frame, a blade attached to the arm and moveable relative to the frame, and a locking mechanism that is moveable from a first position to a second position to prevent movement of the blade in at least two, non-collinear directions. In further embodiments, the locking mechanism is moveable from a first position to a second position to prevent movement of the blade in at least three, non-collinear directions.

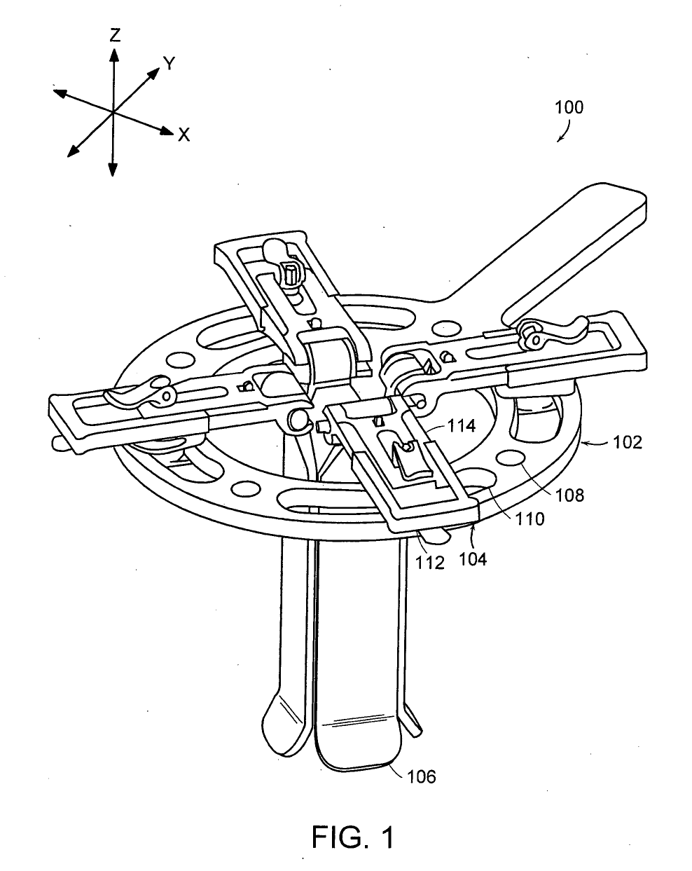

FIG. 1 illustrates a perspective view of an embodiment of the invention that includes surgical retractor 100. Retractor 100 includes a base or frame 102 and a plurality of blade assemblies, each comprising one arm attached or assembled to one blade (e.g., arm 104 and blade 106).

Frame 102 is flat and has a major plane that is coplanar or parallel to the X-Y plane. Frame 102 is circular and includes a plurality of attachment ports 108 and attachment tracts 110 arranged along and within the circumference of frame 102. Ports 108 and tracts 110 provide frame attachment points for surgical instruments or arms.

Tract 110 provides a line of contact between frame 102 and arm 104. Arm 104 is movable in relation to frame 102 along tract 110, thereby allowing movement of at least a portion of an attached blade in a direction that is parallel to the line of contact between arm 104 and frame 102. Arm 104 is fixable at a position along tract 110 by a mechanism described below, thereby allowing arm 104 to be fixed at a point of contact along the line of contact and preventing movement of blade 106 in a direction that runs parallel to tract 110 and/or follows an arc.

Arm 104 has a major axis that is collinear or parallel to the Z axis, normal to the major plane of frame 102, and passes through or near points of contact between arm 104 and frame 102. Arm 104 can rotate about its major axis, thereby moving blade 106 about the points of contact between arm 104 and frame 102 (e.g., in a direction that follows an arc that is coplanar or parallel to the major plane of frame 102).

Arm 104 includes linear slide base 112 and linear slide 114. Linear slide 114 translates into and out of linear slide base 112, thereby moving the blade in a direction that is collinear or parallel to a line that is normal the major axis and parallel or coplanar with the major plane.

Frame 102 is circular in shape. However, in other embodiments, the frame of the invention can be virtually any shape. For example, the frame of the invention can be an ellipse, an oval, a square, or other polygonal shape. In some embodiments, the frame is a completely circular, elliptical, or polygonal shape (i.e., a circular or polygonal shape that is not missing a portion of the circular or polygonal periphery). In other embodiments, the frame is substantially circular, elliptical, or polygonal in shape and defines a partially open or incomplete circular, ellipse, or polygonal shape (i.e., a shape that is basically circular or polygonal, but is missing a portion of a circular or polygonal periphery). In still other embodiments, the frame of the invention is linear in shape. Optionally, frame 102 is adjustably attached to a supporting structure (not illustrated in FIG. 1), such as a table, a rack, a cart, or the like.

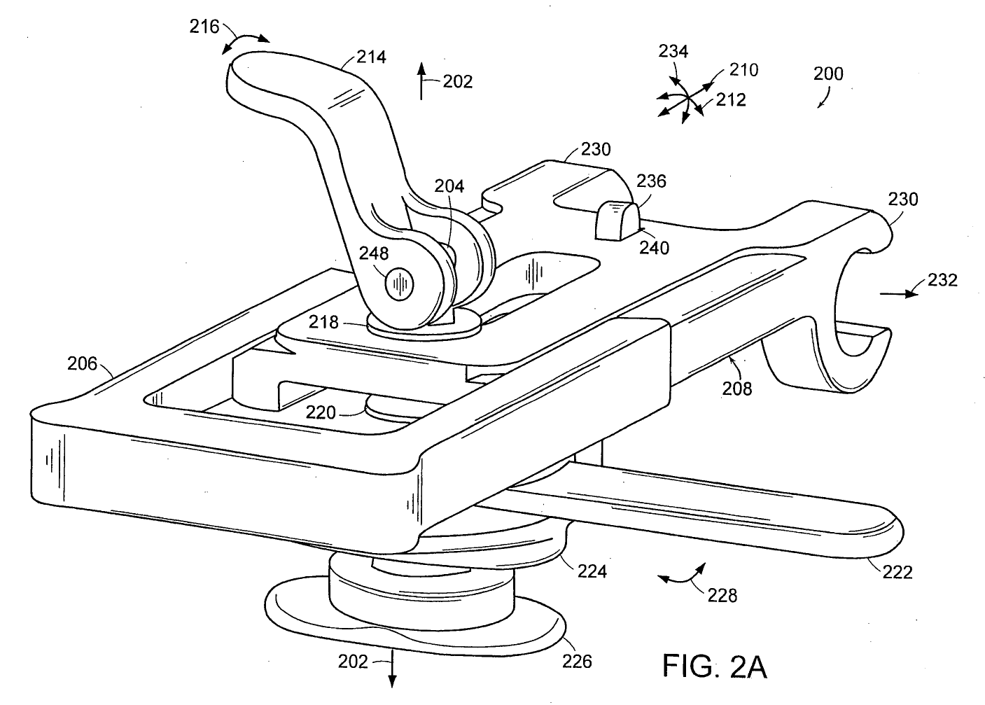

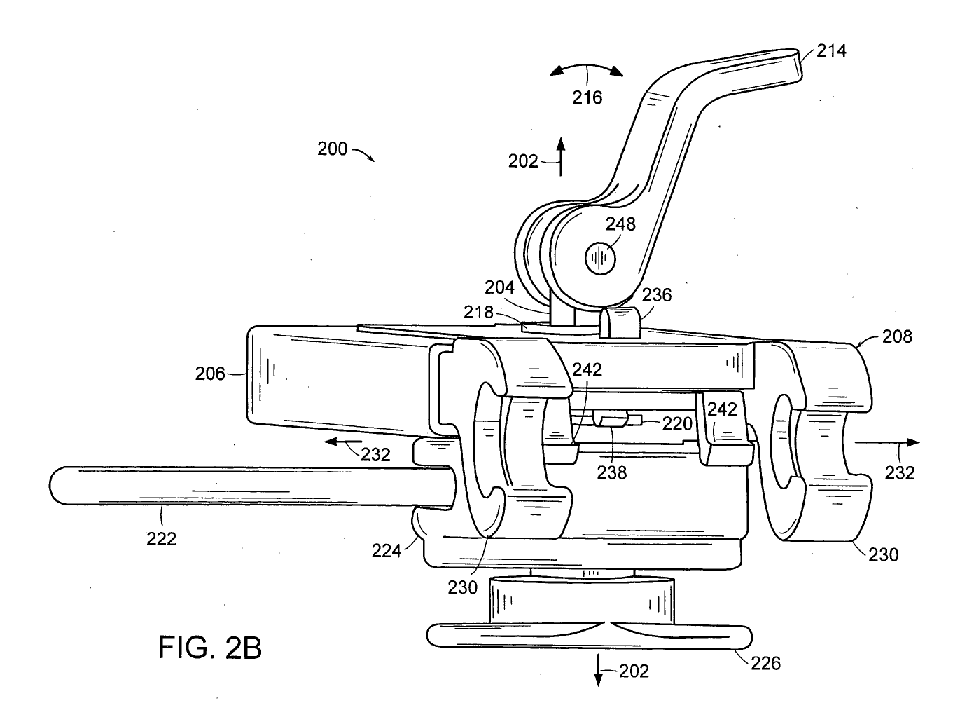

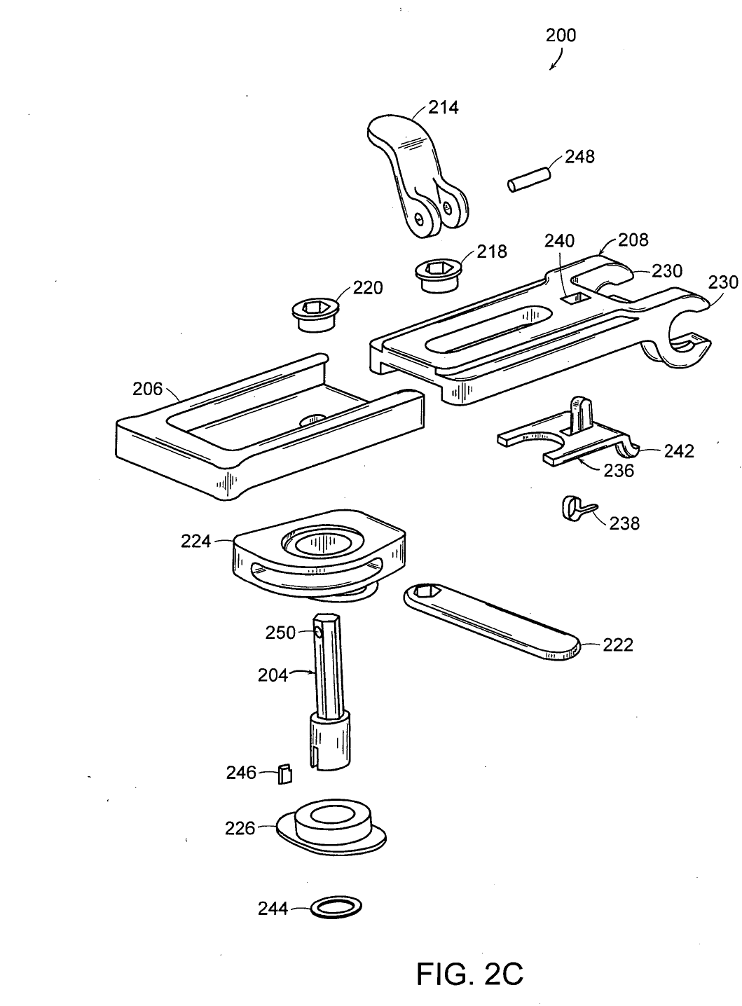

FIGS. 2A and 2B illustrate two different perspective views of a portion of a retractor of the invention that includes arm 200. FIG. 2C illustrates a perspective view of disassembled arm 200. Arm 200 includes major axis 202 which is collinear with the central axis of axel 204. When assembled to a frame, major axis 202 extends through a point of contact (or region between points of contact) between arm 200 and the frame and is normal to the major plane of the frame.

Arm 200 includes linear slide base 206 and linear slide 208. At least a portion of linear slide 208 extends into linear slide base 206. Linear slide 208 can move or translate in relation to linear slide base 206 along direction 210 of arm 200, thereby allowing a practitioner of the invention to translate an attached surgical blade along direction 210. Direction 210 extends along a line that is normal to major axis 202. When assembled to a frame, major axis 202 extends through points or regions of contact between arm 200 and the frame and is collinear or parallel to direction 210.

Linear slide base 206 and linear slide 208 can also rotate about axis 202 along direction 212, thereby allowing a practitioner of the invention, when arm 200 is assembled to a frame and a blade, to move at least a portion of the blade relative to the frame in a direction that extends about a point of contact between arm 200 and a frame, is coplanar or parallel to the major plane of the frame, and its axis of rotation is normal to direction 210. When linear slide 208 has been translated to a desired position relative to base 206 and/or rotated to a desired position about major axis 202, linear slide locking lever 214 can be moved from a first position to a second position along line 216, thereby pressing washer 218 downward against slide 208 and washer 220, fixing the position of slide 208 with respect to base 206 and/or major axis 202, and preventing movement of linear slide 208 (and any attached blades) along direction 210 or about points of contact between arm 200 and an attached frame. FIGS. 2A and 2B illustrate lever 214 in the first position (i.e., the position that allows slide 208 to move relative to base 206 and/or major axis 202). As a comparison, the embodiment of the invention illustrated in FIG. 1 includes linear slide locking levers in a second position (i.e., the position that prevents linear slide 114 from moving relative to base 112 or rotating about an axis that is perpendicular to the plane of the frame with an origin located at the point of contact between frame 102 and arm 104).

Arm 200 includes lever 222 and frame attachment washers 224 and 226. Attachment washers 224 and 226 assemble or attach arm 200 to a frame of a retractor of the invention. Attachment washer 226 is asymmetrical, being in the shape of an ellipse with the major axis of the ellipse being greater than the width of an attachment point or tract, and the width of the minor axis of the ellipse being smaller than the width of the attachment point or track. Lever 222 and attachment washer 224 are both fixed and rotatably attached to axel 204 so that when lever 222 is moved in direction 228 and about major axis 202 attachment washer 226 also rotates about axis 202.

To attach arm 200 to a frame, the minor axis of lever attachment washer 226 is aligned with an attachment track and washer 226 is directed through the track to the bottom side of the frame. Attachment washer 224 is located above washer 226 and sits on the top side of the frame and partly extends into the track. A practitioner of the invention pushes lever 222 along direction 228, thereby rotating axel 204 and washer 226 clockwise and preventing washer 226 from emerging from the frame.

Washer 226 is non-uniformly thick around the circumferences of the elliptical portion so that rotating lever 222 causes washers 224 and 226 to press against a frame with varying degrees of force depending upon the extent to which lever 222 is rotated. For example, rotating lever 222 in a clockwise direction about axis 202 can cause increasingly thicker portions of washer 226 into contact with a frame, thereby pressing washers 224 and 226 to press against the frame with an increasing amount of force until movement of arm 200 along the attachment track is completely impeded by the resulting friction, while rotating lever 222 in a counter-clockwise direction decreases the impedance between washers 224 and 226 and the frame and/or positions washer 226 so that arm 200 can be disengaged from the track. In this manner, a practitioner can slidably attach arm 200 to a frame and/or fix the position of arm 200 at a position about the circumference of a frame.

Arm 200 includes blade attachment points 230 where a retractor blade can be rotatably attached to arm 200. A blade attached to arm 200 at points 230 is rotatable about axis 232 and along direction 234. Axis 232 is normal to direction 210 and, when arm 200 is attached to a frame, is coplanar or parallel to a major plane of the frame. Direction 234 extends about a point of contact between arm 200 and the frame and is normal to directions 210 and 212.

Arm 200 also comprises a locking mechanism that includes blade rotation lock plate 236 and tension spring clip 238. A portion of plate 236 is located below slide 208, and a portion of plate 236 extends through hole 240. Hole 240 is defined by slide 208. Plate 236 includes one or more teeth 242. Spring clip 238 presses against plate 236 and causes teeth 242 to engage a portion of an attached blade, thereby locking the attached blade in a desired position about axis 232. Depressing the portion of plate 236 that extends through hole 240 disengages teeth 242 from an attached blade, thereby allowing a practitioner to rotate the blade along direction 234 and to a desired position about axis 232.

Axel locking washer 244 is attached to the bottom end of axel 204 and secured thereto by block 246. Pin 248 extends through hole 250 and attachment holes defined by lever 214, thereby securing lever 214 to axel 204.

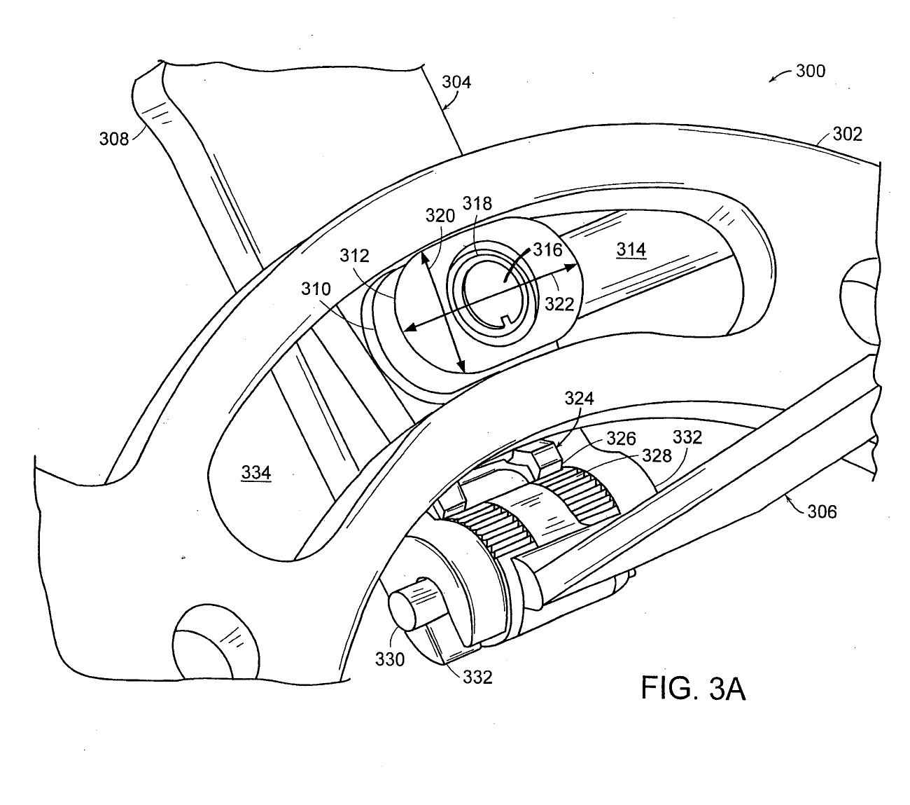

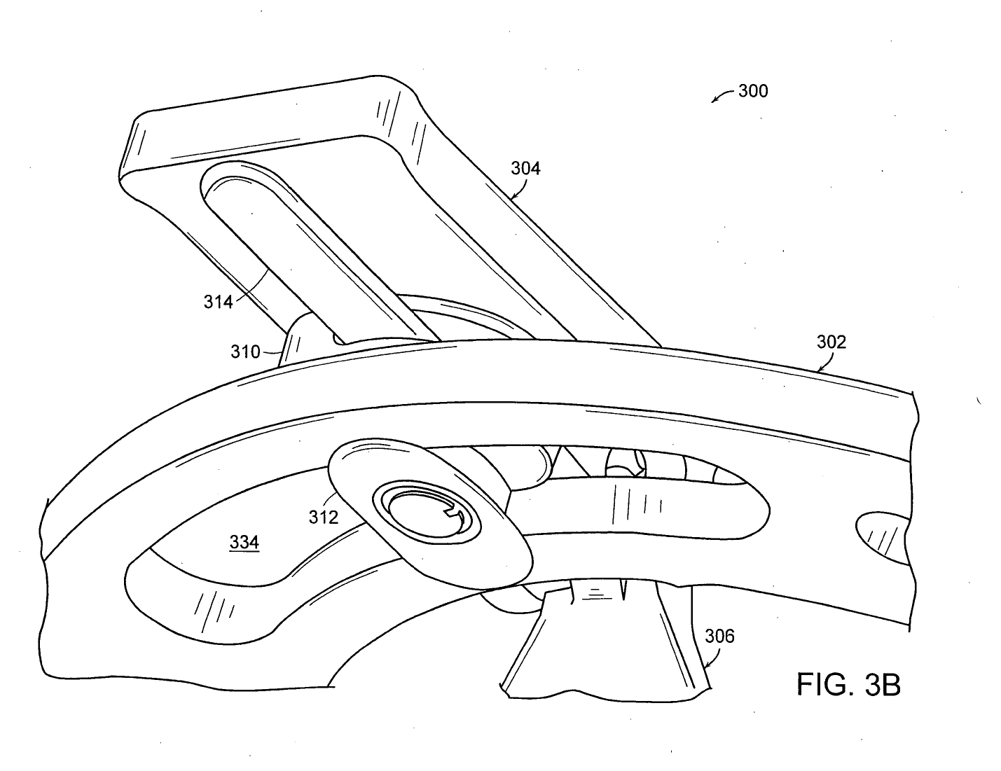

FIGS. 3A and 3B include two perspective views from different angles of a bottom or ventral portion of an embodiment of the invention that includes retractor 300 and that illustrates an arm attached to a frame. Retractor 300 includes frame 302 and a blade assembly that includes arm 304 and blade 306 attached or assembled to arm 304.

Arm 304 comprises linear slide base 308, frame attachment washer 310, frame attachment washer 312, and lever 314 all assembled to axel 316 and secured thereon by axel locking washer 318. In some embodiments, attachment washer 312, axel 316, and/or lock washer 318 are formed from a single portion of plastic, metal, or other construction material. Arm 304 also includes blade rotation lock plate 324 which has a plurality of teeth 326 which are engaged in a complementary manner with teeth 328. Frame attachment washer 312 has short axis 320 and long axis 322.

Blade 306 is attached or assembled to arm 304 with attachment pin 330 which extends through blade attachment points 332 and a lumen defined by the distal portion of blade 306.

Frame 302 includes attachment track 334 to which arm 304 is attached. FIG. 3A illustrates lever 314 in a first position. In this first position, short axis 320 of washer 312 is aligned with the short axis of washer 310 to facilitating the attachment or assembly of arm 304 to frame 302 (i.e., axis 320 is at a normal angle to the circumference of frame 302 and long axis 322 is roughly parallel or collinear with the circumference of frame 302). Short axis 320 is sufficiently narrow to allow washer 312 to be directed through attachment track 334 and to the bottom side of frame 302.

Once washer 312 is positioned on the bottom side of frame 302, lever 314 is rotated to another position, thereby rotating washer 312 and causing some portion of long axis 322 to overlap with a portion of frame 302 that defines track 334. In other words, the rotation of washer 312 causes short axis 320 to be positioned to the circumference of frame 302 at some angle other than normal. The resulting overlap prevents arm 308 from disengaging from frame 302.

Washer 312 is non-uniformly thick around the elliptical circumference. That is, the distance separating a point on the bottom surface of washer 312 and a corresponding point on the top surface of washer 312 is greater at the portion of the circumference at either ends of long axis 322 than at the portion of circumference at either ends of short axis 320. Rotating lever 314 and washer 312 causes different portions of this non-uniformly thick circumference to come into contact with the bottom surface of the portion of frame 302 that defines track 334. When lever 314 is in the first position (i.e., the position illustrated in FIG. 3A), no portion of washer 312 is forcibly contacting the portion of frame 302 that defines track 334 and arm 308 can be freely removed from contact with frame 302. When lever 312 is in another position (e.g., the position illustrated in FIG. 3B), the thickest portion of the circumference of washer 312 (i.e., the portion around the circumference at either end of long axis 322) is contacting the portion of frame 302 that defines track 334, thereby causing washers 312 and 310 to press against frame 302 and preventing movement of arm 304 along track 334.

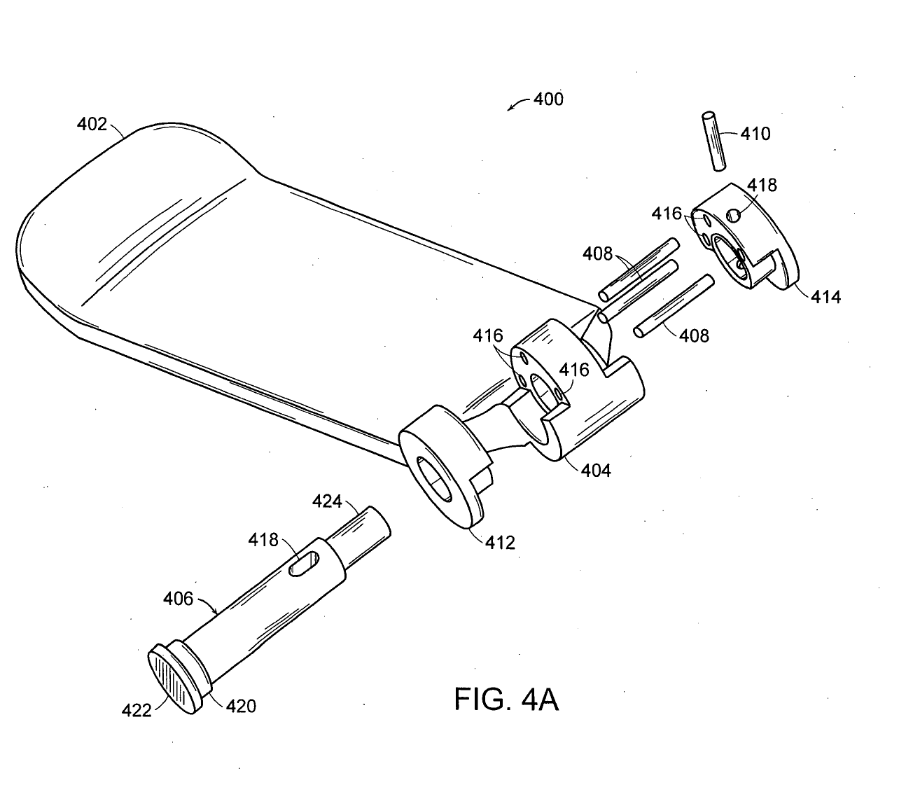

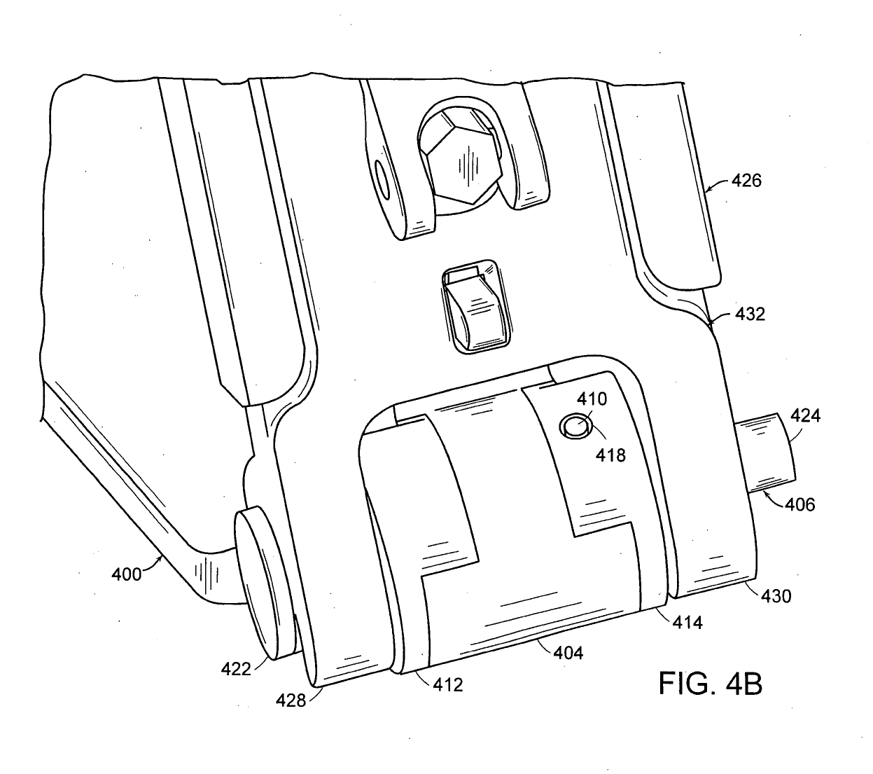

FIG. 4A illustrates a portion of an embodiment of the invention that includes disassembled blade 400. Proximal end 404 includes blade attachment pin 406, pins 408, pin 410, and blade attachment washers 412 and 414. When blade 400 is fully assembled, pins 408 extend through holes 416 and secure washers 412 and 414 on either side of proximal end 404. Blade attachment pin 406 includes interference ring 420 at end 422 and pin hole 418 near end 424. Pin 406 extends through washers 412, 414 and end 404 and is held in place by pin 410 which extends through holes 418. In some embodiments, washers 412, 414, pins 408, end 404, and/or the blade are manufactured from a single portion of plastic, metal, or other construction material.

FIG. 4B illustrates blade 400 attached or assembled to arm 426. Pin 406 secures blade 400 to attachment points 428 and 430.

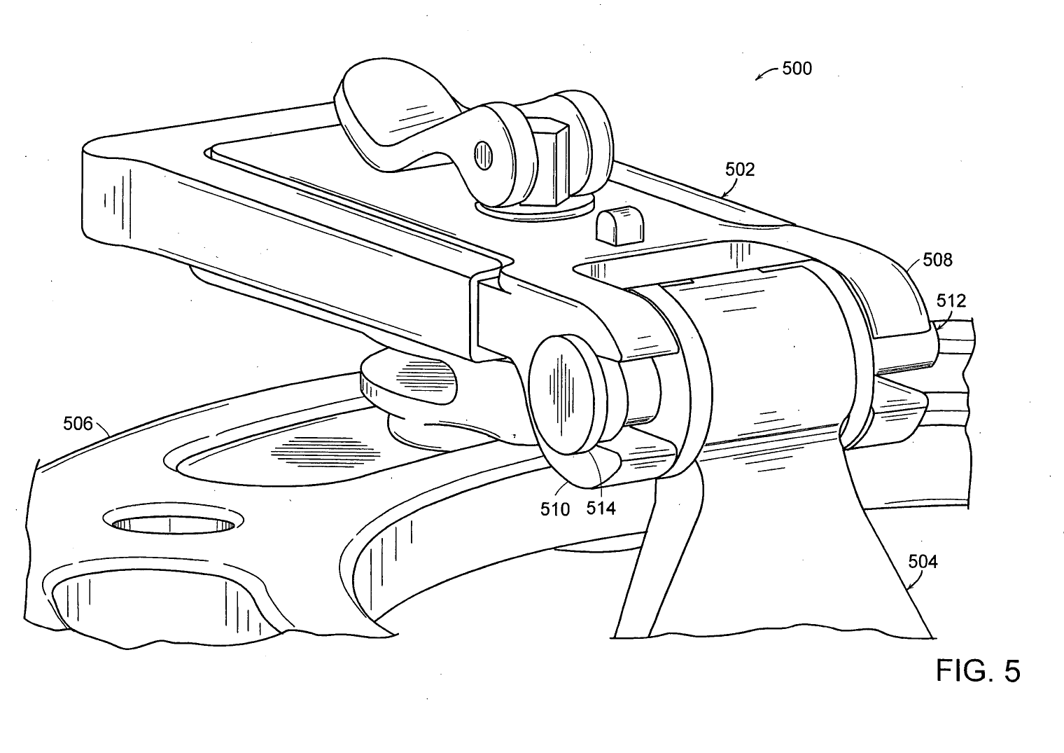

FIG. 5 illustrates an embodiment of the invention that includes a portion of retractor 500. Retractor 500 includes frame 506 and a blade assembly comprising arm 502 and blade 504. Arm 502 includes blade attachment point 510 and blade attachment point 508. Blade 504 includes pin 512 having interference ring 514. Pin 512 extends through blade attachment points 510 and 508, thereby securing blade 504 to arm 502. Interference ring 514 produces an interference fit between pin 512 and blade attachment point 510, thereby preventing blade 504 from disengaging arm 502.

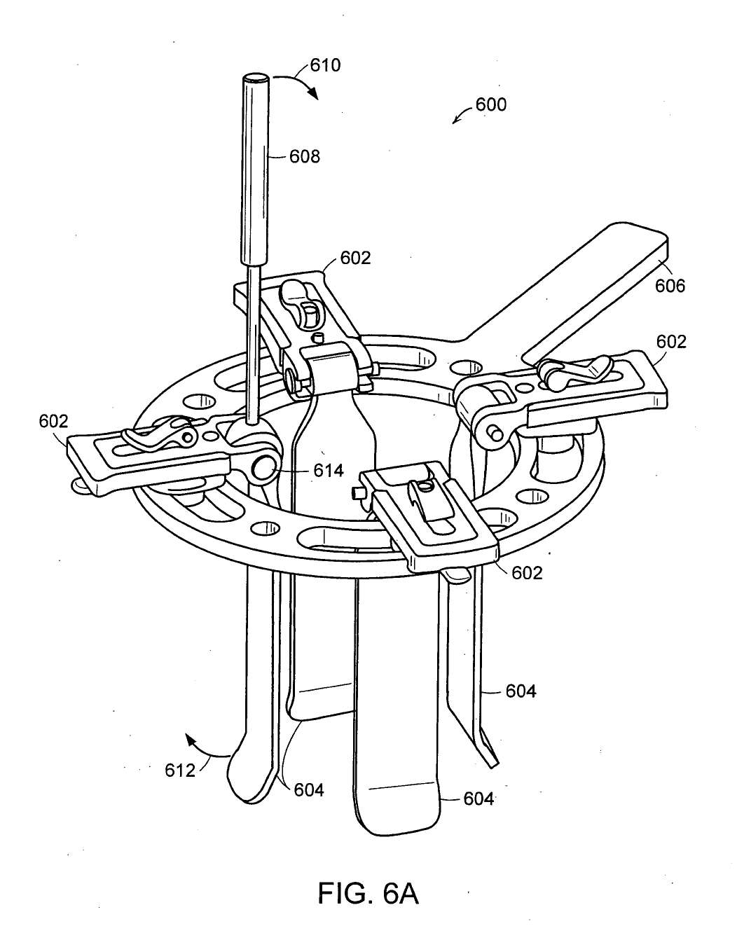

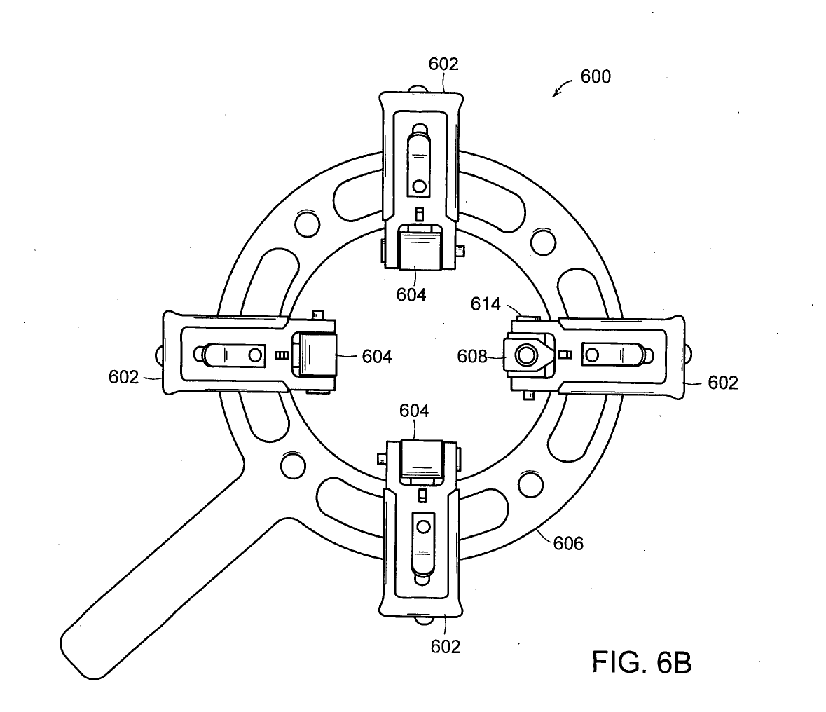

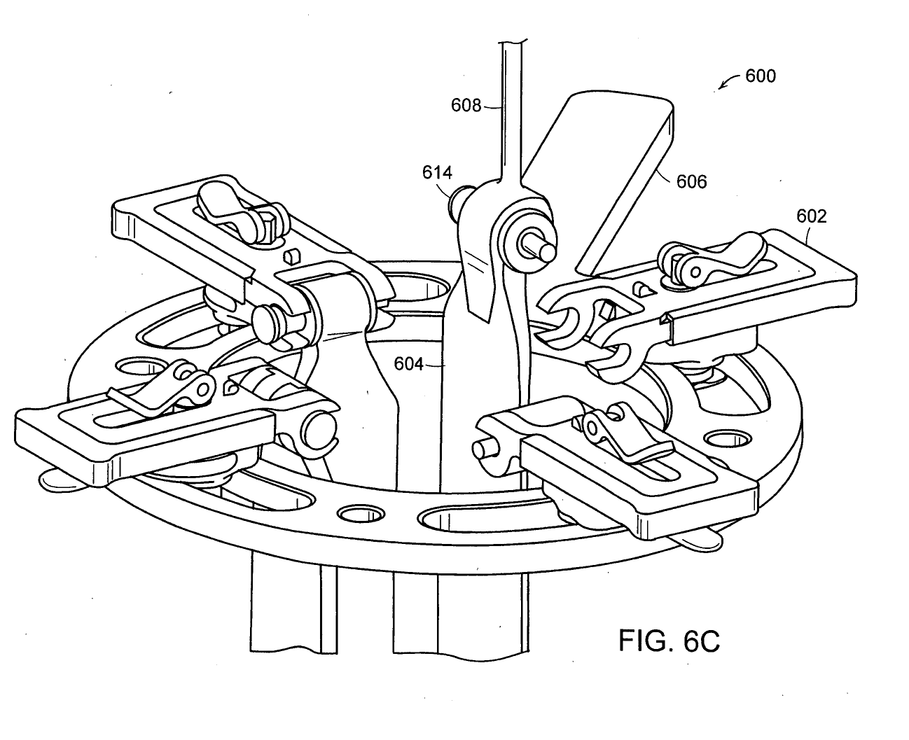

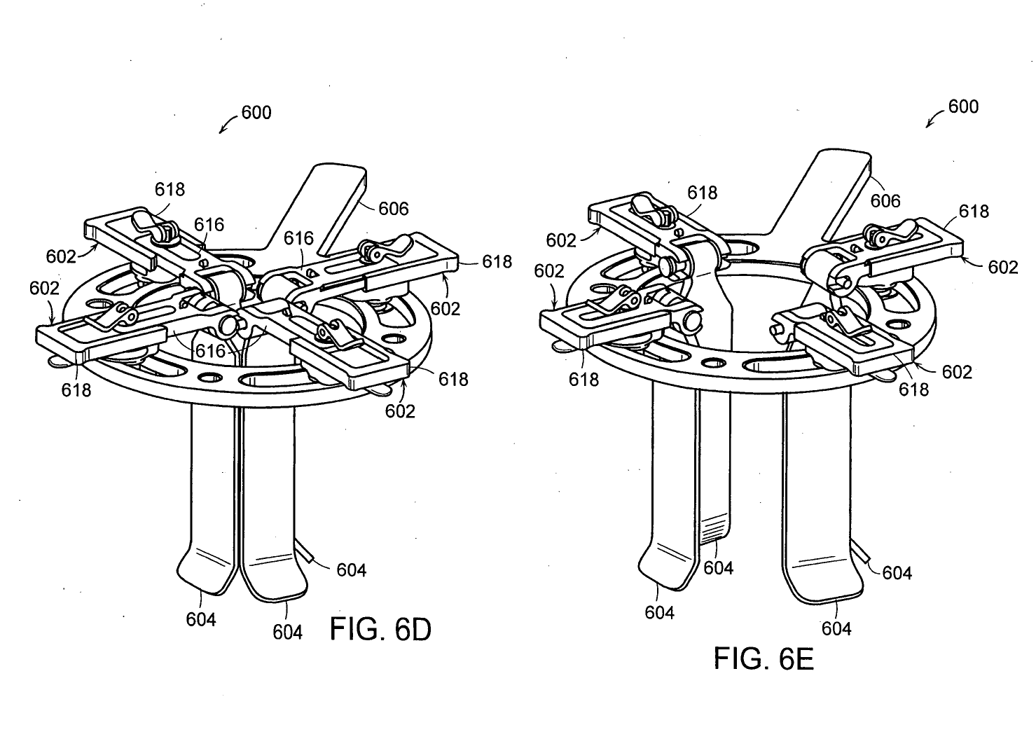

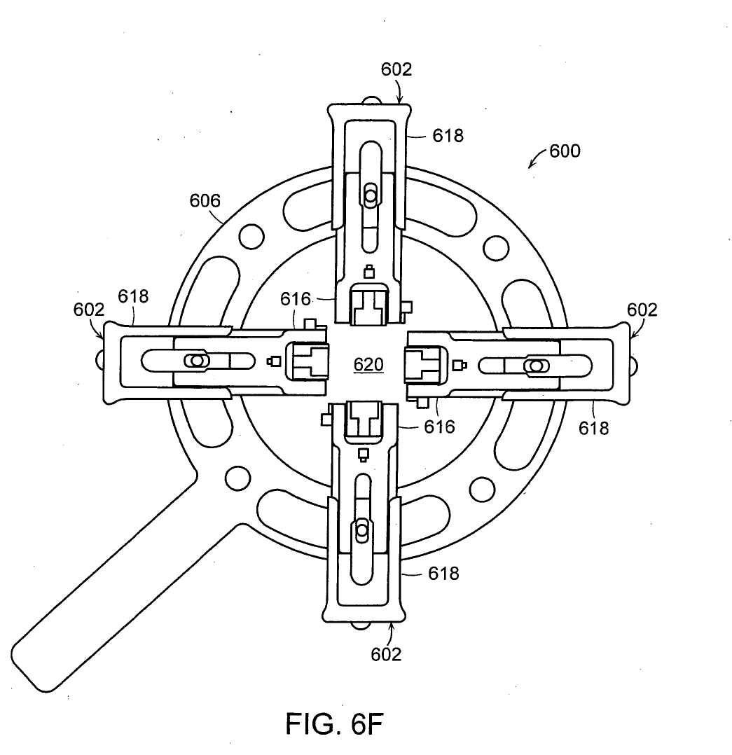

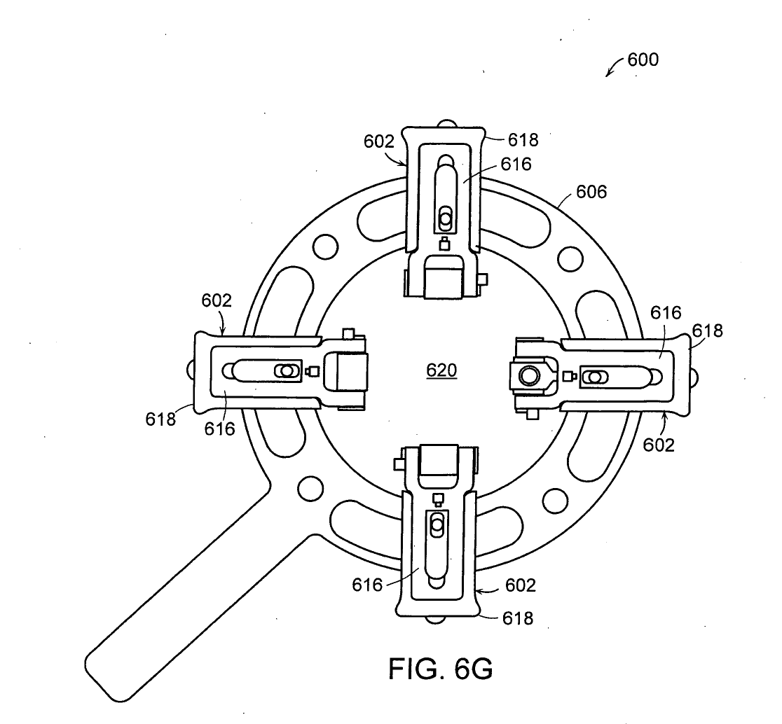

FIG. 6A illustrates a perspective view of an embodiment of the invention that includes a retractor 600. Retractor 600 comprises frame 606 and plurality of blade assemblies that include arms 602 and blades 604. Blade attachment and rotation tool 608 is assembled to blade 604 and is used to attach blade 604 to, or remove blade 604 from, arm 602. Tool 608 is also useful for rotating blade 604 about pin 614. A practitioner of the invention directs tool 608 along direction 610, thereby rotating blade 604 along direction 612 and about a line that is coplanar or parallel to a major plane of frame 606 and normal to the major axis of arm 602. FIG. 6B illustrates a plane view of retractor 600 that is shown in FIG. 6A.

FIG. 6C illustrates another perspective view of retractor 600. Tool 608 is attached to blade 604. Tool 608 is used to attach blade 604 to, or remove blade 604 from, arm 602.

FIGS. 6D and 6E illustrate perspective views of retractor 600 with retractor blades 604 retracted or contracted, respectively. FIGS. 6F and 6G illustrate plane view of retractor 600 that are shown in FIGS. 6D and 6E, respectively.

In FIGS. 6D and 6F, linear slides 616 are partially extended out from linear slide bases 618 and into the central portion of area 620 of frame 606. Retractor blades 604 are in a contracted position and are in relatively close proximity to each other. This contracted position is useful for inserting or positioning retractor 600 in a mammalian body because the contracted blades minimize the length of the incision that is needed to introduce or position the retractor within the mammal.

In FIGS. 6E and 6G, linear slides 616 are situated within bases 618 and blades 604 are retracted to the peripheral portion of area 620 of frame 606. Retractor blades 604 are relatively distal from each other. This retracted position is useful for creating a window or access way to a surgical site within the mammal.

While FIGS. 6D-6G illustrate retractor blades 604 in two positions, it will be recognized that extending or retracting one or more of slides 616 to various extents relative to the respective base 602 will allow a practitioner to position the blades in an almost infinite number of arrangements. In this manner, the practitioner can position blades 604 to create a desired working space within a mammal for a surgical procedure.

While FIG. 6A illustrate retractor blades that are essentially flat along a majority of the length of the blades, in other embodiments of the invention, the retractor blades are curved or arcute along at least a portion of a major axis that extends from the proximal end to the distal end of the blade and/or do not have a bent or angled portion on the distal end.

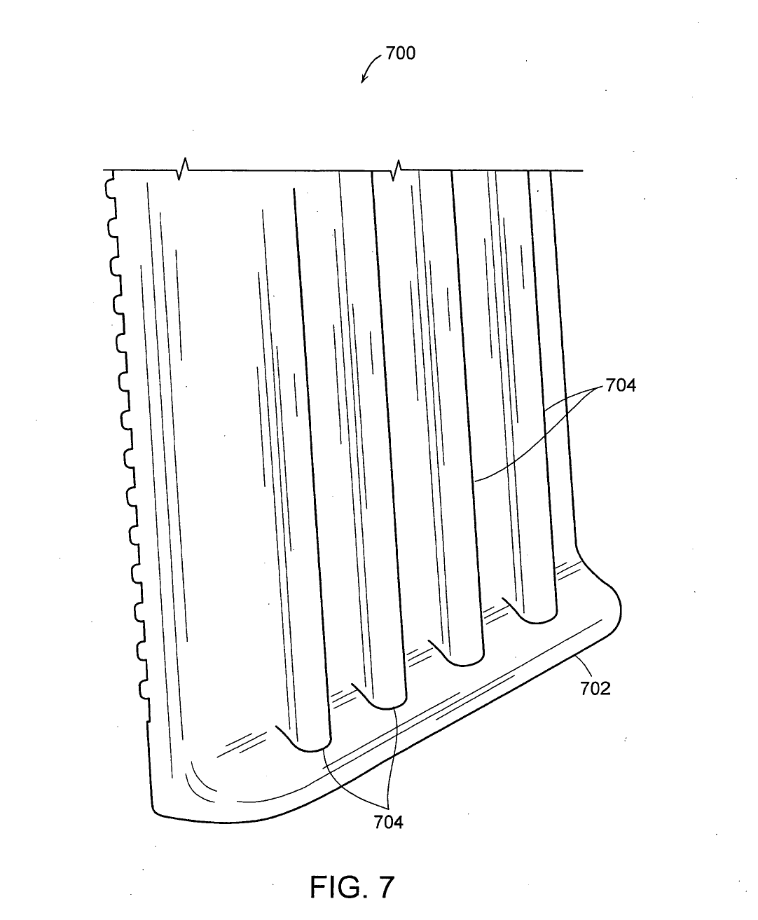

Optionally, one or more blades of a retractor of the invention include a toe-out protrusion extending from the distal end of the outer face. FIG. 7 illustrates a close up view of the distal end of the outer face of blade 700. Toe-out protrusion 702 extends from the outer face of blade 700. Toe-out protrusion 702 allows blade 700 to more effectively retract tissue from a surgical site compared to a similar blade extension lacking a toe-out protrusion. In further embodiments, the toe-out protrusion extends at an angle from the face of the blade at an angle that is greater or lesser than 90°. Blade 700 also includes ridges 704 which extend at least a portion of the length of blade 700, and provide more effective retraction of tissue from a surgical site compared to a similar blade lacking such ridges.

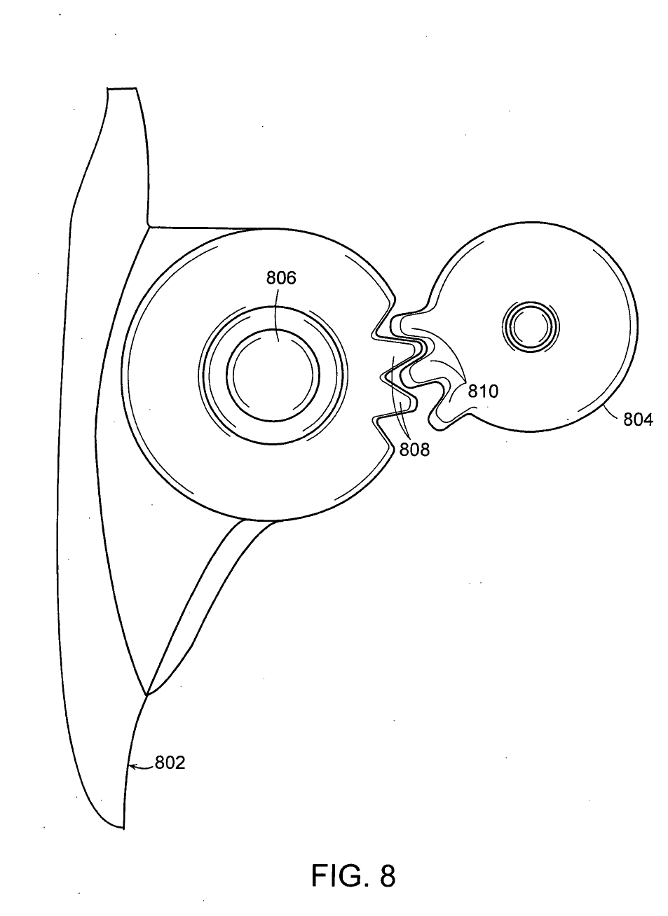

FIG. 8 illustrates a side view of a portion of blade 802 that demonstrates the interaction between a blade and a portion of an arm that includes spurred gear 804. Blade 802 includes attachment pin 806 and a plurality of teeth 808. Gear 804 includes a plurality of teeth 810. Teeth 808 and teeth 810 are complementary and provide for fixable rotation of blade 802 about a direction that extends circumferentially about a line that is normal to the major axis of the arm and parallel to the major plane of the frame.

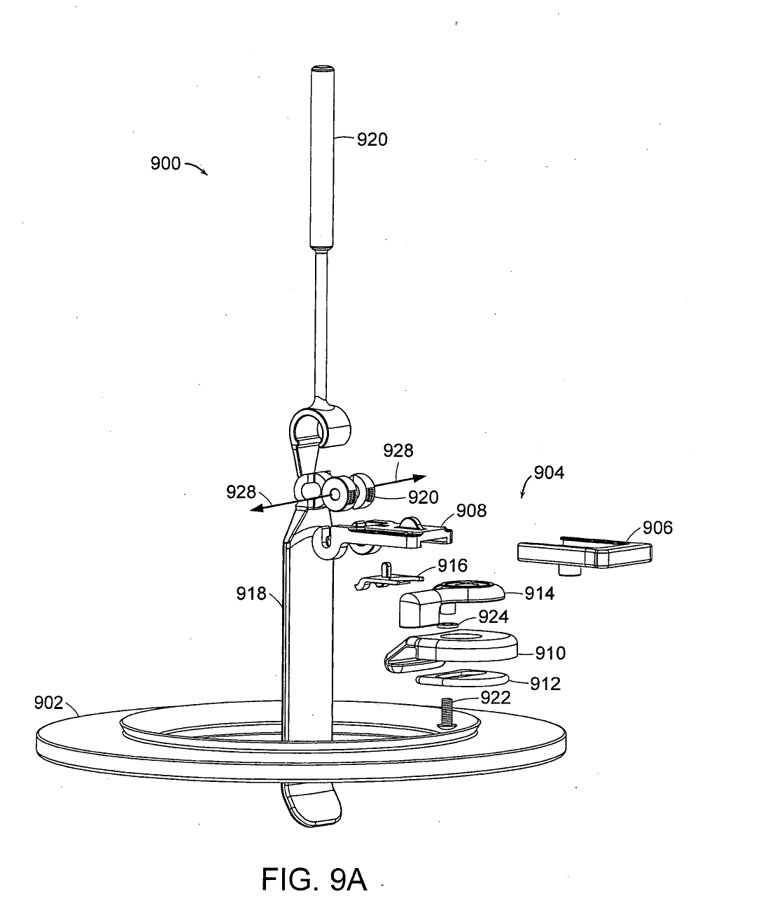

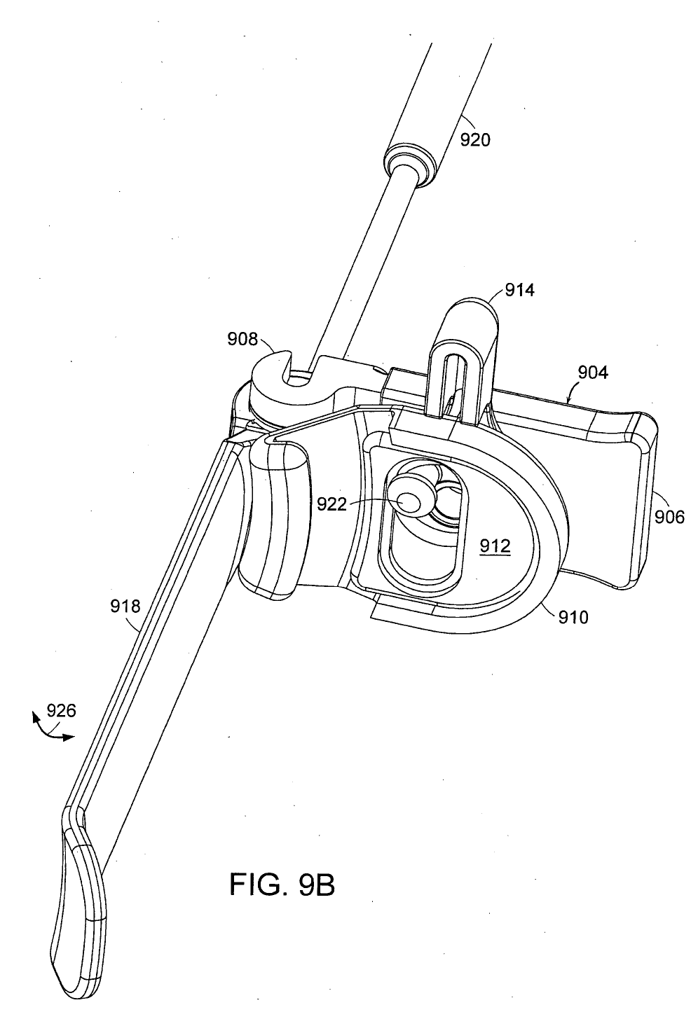

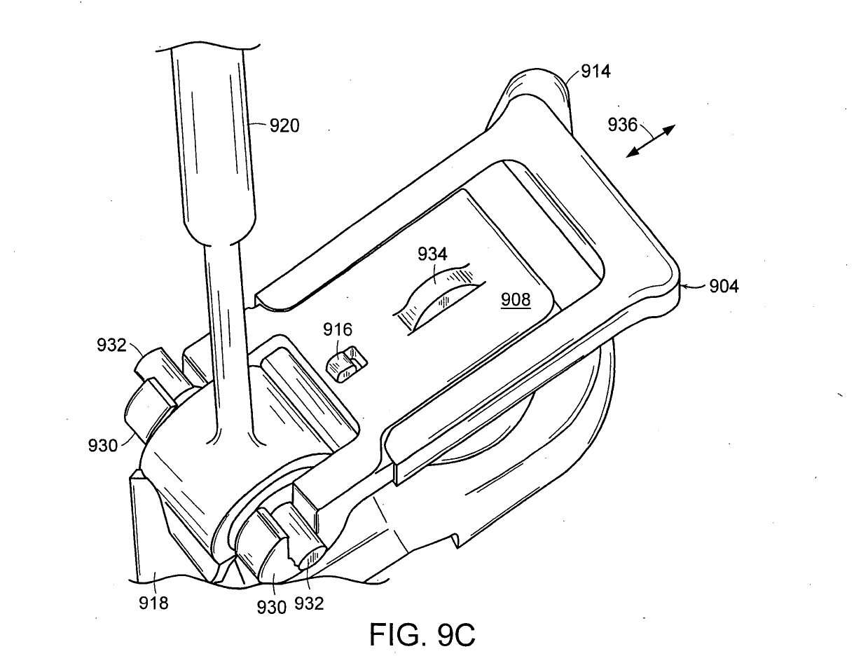

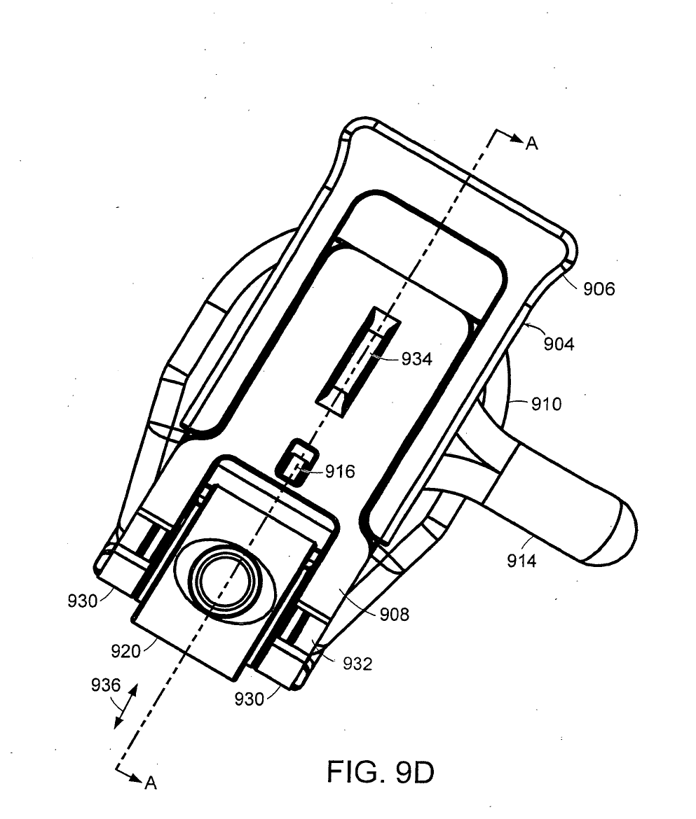

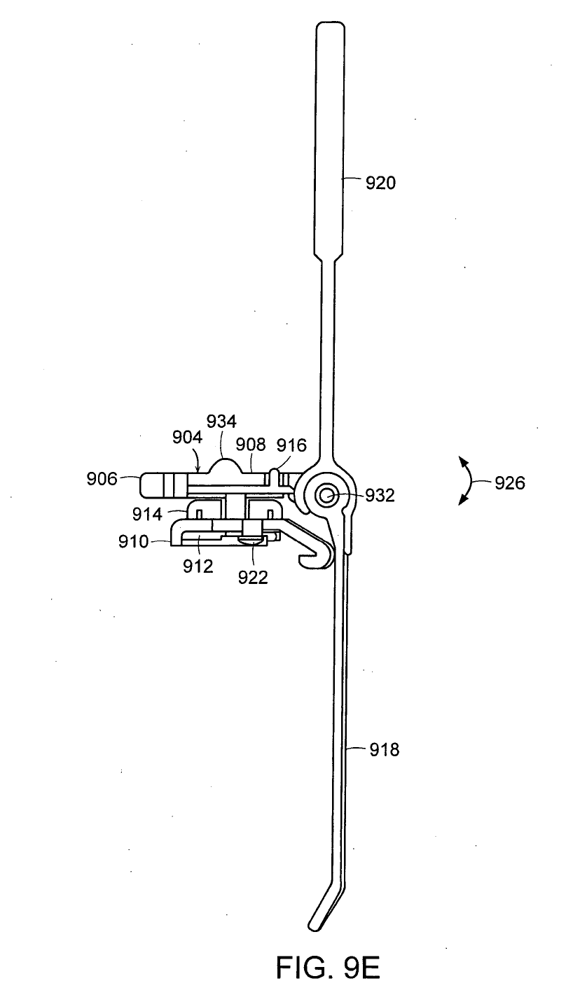

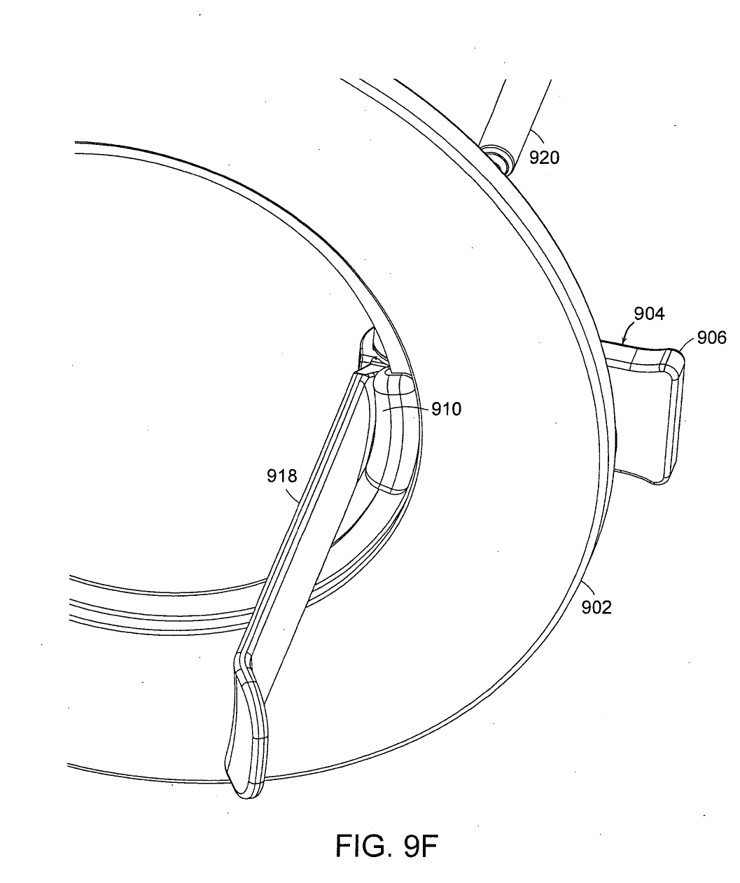

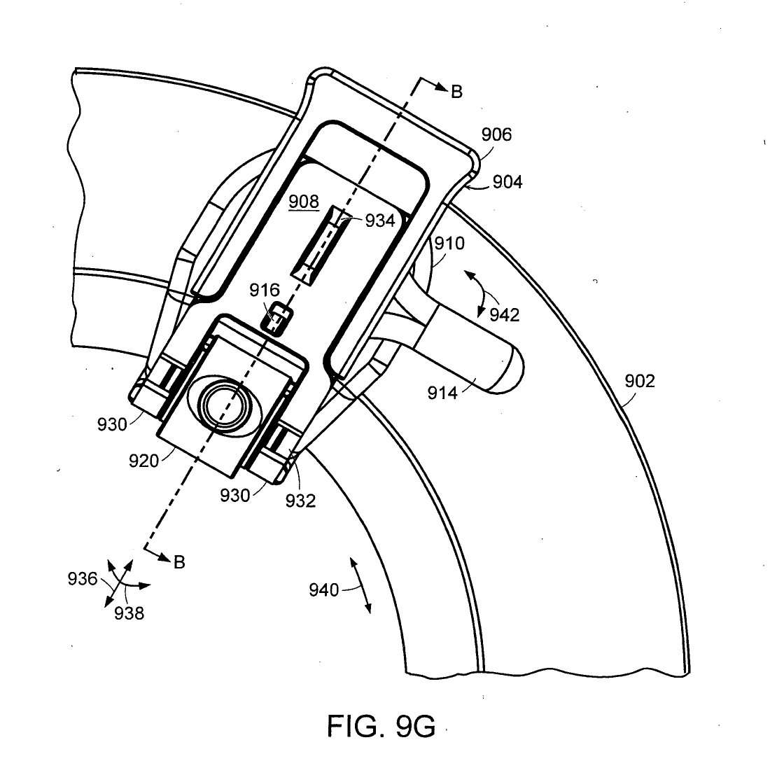

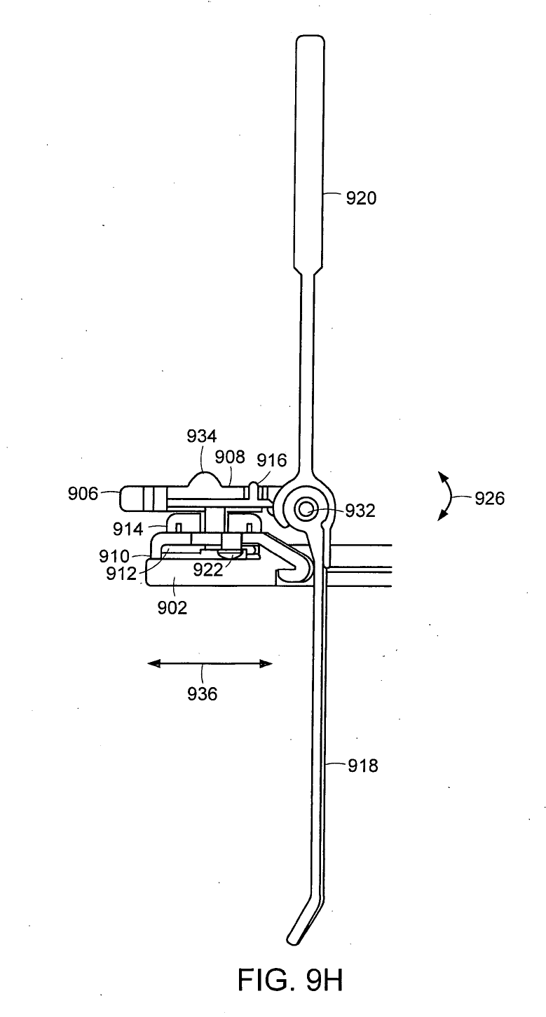

FIG. 9A illustrates retractor system 900 of the invention that includes frame 902 and a dissembled view of retractor arm 904. Retractor arm 904 includes linear slide base 906, linear slide 908, frame attachment plates 910 and 912, arm locking lever 914, and blade rotation lock plate 916.

FIGS. 9B-9E illustrate various views of assembled arm 904 and blade 918. FIG. 9B illustrates a posterior view of assembled arm 904 and blade 918. Locking screw 922 extends through an orifice defined by plate 912, through an orifice defined by plate 910, through washer 924, and into a complementary screw attachment hole defined by lever 914. Blade 918 is assembled to linear slide base 908. Blade attachment tool 920 can be used to assemble blade 918 to arm 904. Plate 916 (not illustrated in FIG. 9B) of arm 904 can engage teeth 920 of blade 918, thereby adjustably fixing blade 918 at a desired position along direction 926 and about axis of rotation 928 (illustrated in FIG. 9A).

FIGS. 9C and 9D illustrate two views of the top of assembled arm 904 and blade 918. FIG. 9E illustrates an interior view of a cross-section of assembled arm 904 and blade 918 along line A of FIG. 9D. Arm 904 includes a pair of blade attachment points 930. Blade 918 includes blade attachment pin 932 (not illustrated in FIGS. 9A and 9B). The two ends of pin 932 are directed into the complementary slots in attachment points 932.

Linear slide 908 includes protrusion 934 which can be used to direct linear slide along direction 936. A practitioner of the invention applies pressure to protrusion 934 to move slide along direction 936, thereby moving blade 918 along a direction that is normal to a major axis of arm 904 and parallel or coplanar to a major plane of frame 902. Arm 904 includes a ball plunger mechanism (not illustrated in the Figures) to secure the position of slide 908 relative to blade 918 along direction 936. Slide 908 includes a ball plunger that is assembled to a spring plate and is complementary to a series of grooves or detents that line the inner surface of base 906. The spring plate cause the plunger to presses against the detent track. A practitioner of the invention depresses the plate to release the plunger and allow slide 908 (and any attached blade) to translate along direction 936 relative to base 906.

FIG. 9F illustrates a view of the bottom of assembled arm 904 attached to frame 902. FIG. 9G illustrates a view of the top of assembled arm 904 attached to frame 902. FIG. 9H illustrates an interior view of a cross-section of assembled arm 904 attached to frame 902 along line B of FIG. 9G. A practitioner of the invention can position arm 904 and blade 918 at virtually any point along direction 940 on frame 902.

Arm 904 and blade 918 can be rotated about a major axis that is normal to a major plane of frame 902 and extends through or between points of contact between arm 904 and frame 902. Plate 912 is slightly curved so that moving lever 914 along direction 942 from a first position (e.g., the position illustrated in FIG. 9G) to a second position (e.g., the position illustrated in FIG. 9C) causes plate 912 to press against frame 902, thereby securing the relative position of slide 908 and blade 918 along direction 940. In addition, the top or dorsal surface of lever 914 is angled, tapered, or slanted which allows a practitioner of the invention to secure the relative position of slide 908 and blade 918 along direction 938. A practitioner of the invention moves lever 914 from a first position to a second position, thereby causing the tapered top surface of lever 914 to press against the ventral or bottom side of slide base 906. By varying the degree that lever 914 is turned, a practitioner of the invention can control how much force is applied to base 906 by lever 914, and consequently, vary the amount of force needed to rotate an attached blade about an axis that is normal to a major plane of frame 902 and passes through or between points of contact between arm 904 and frame 902 (i.e., along or parallel to direction 938). In this manner, retractor 900 includes a locking mechanism that is moveable from a first position to a second position to prevent movement of the blade in at least two, non-collinear direction.

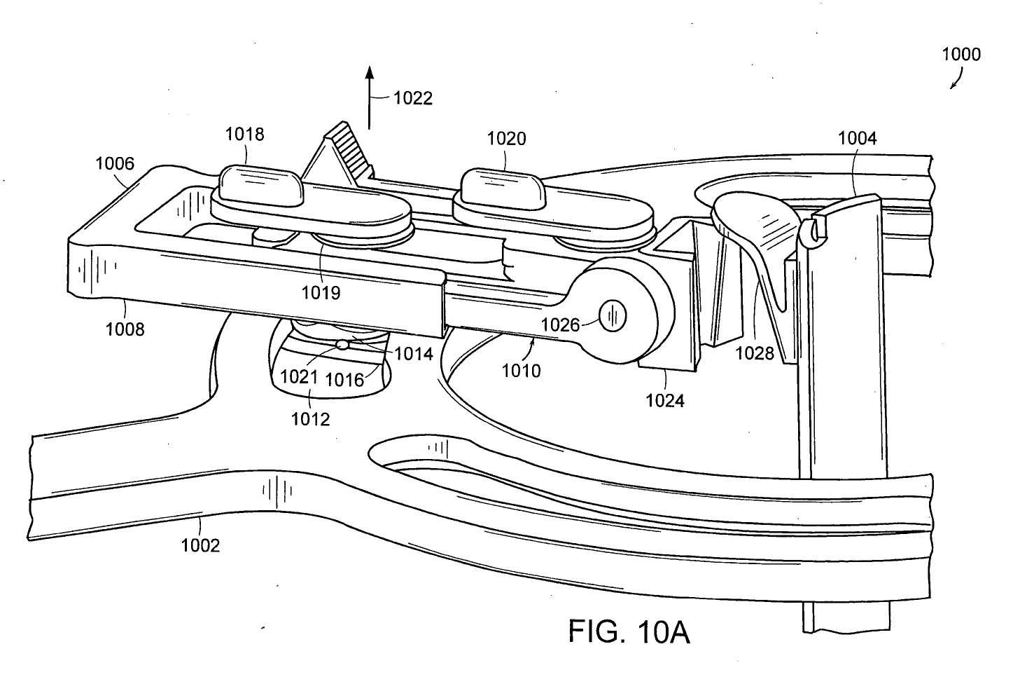

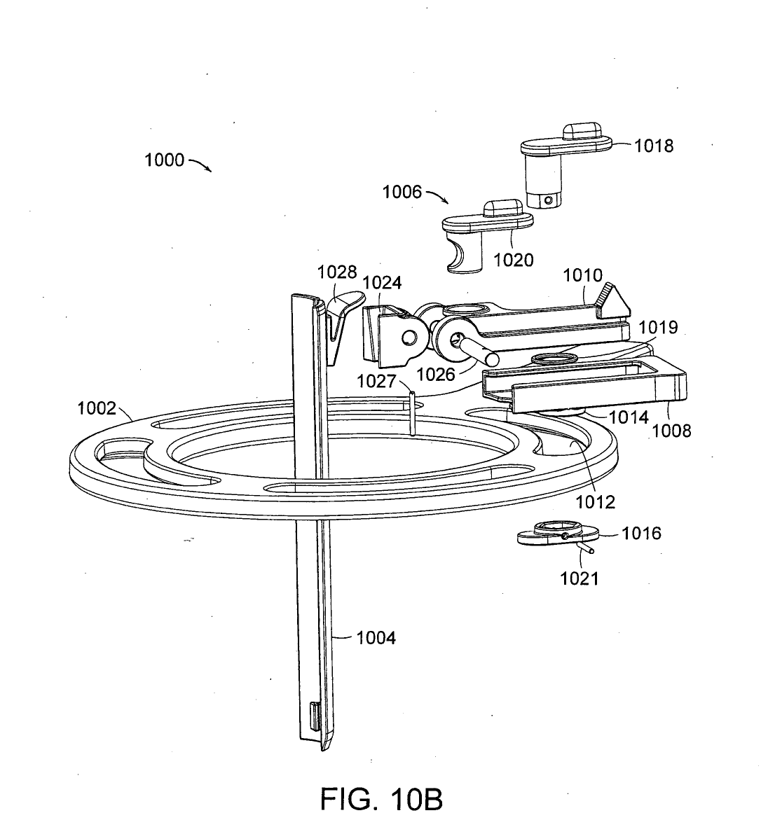

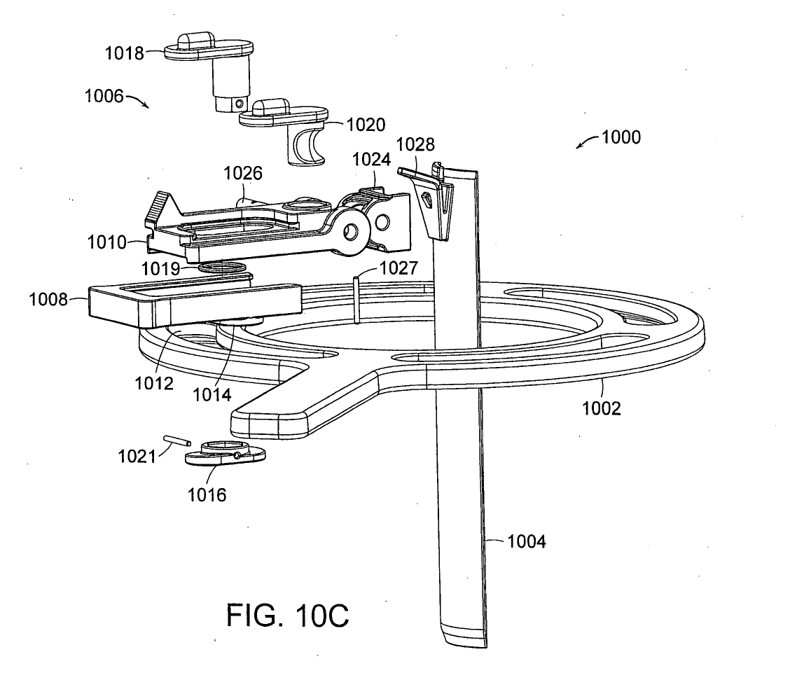

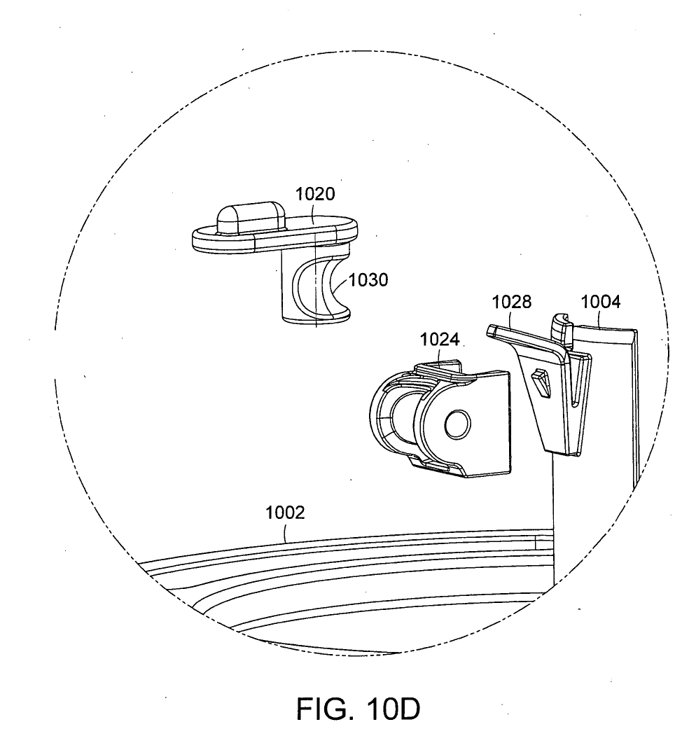

FIG. 10A illustrates another embodiment of the invention that includes a perspective view of a portion of retractor 1000. Retractor 1000 includes frame 1002 and a blade assembly comprising retractor blade 1004 and arm 1006. Frame 1002 includes a plurality of arm attachment tracks 1012. Arm 1006 includes linear slide base 1008 and linear slide 1010. FIGS. 10B and 10C illustrate two views of retractor 1000 with arm 1006 in a dissembled state.

Linear slide base 1008 includes a frame attachment portion 1014 extending from the bottom or ventral side of base 1008. Frame attachment portion 1014 is adapted to extend into frame attachment track 1012. Arm 1006 includes frame attachment plate 1016 which is used to assemble arm 1006 to frame 1002 and, in conjunction with frame attachment portion 1014, lock or fix the position of arm 1006 at a desired location along attachment track 1012.

Arm 1006 includes a locking mechanism that comprises lever 1018. Lever 1018 is assembled to washer 1019, linear slide 1010, slide base 1008, and frame attachment plate 1016. Pin 1021 secures lever 1018 to plate 1016. Turning lever 1018 engages the locking mechanism and fixes the position of blade 1004 in at least two non-collinear directions. For example, turning lever 1018 can fix the position of blade 1004 in a direction that runs parallel to the circumference of the curved portion of frame 1002. In addition, turning lever 1018 can fix the position of slide 1010 relative to base 1008, thereby fixing the position of blade 1004 in a direction that is normal to central axis 1022 of arm 1006 and parallel or coplanar with the major plane of frame 1002.

Arm 1006 includes a locking mechanism that comprises blade rotation locking lever 1020. Lever 1020 is attached to the distal end of slide 1010 near blade attachment slot 1024. Blade attachment slot 1024 is attached to the distal end of slide 1010 by pins 1026 and 1027. Blade 1004 includes a butterfly attachment clip 1028 which is used to attach blade 1004 to slot 1024 of arm 1006. Slot 1024 complements clip 1028. Blade 1004 can be detached from arm 1006 by depressing clip 1028.

Blade attachment slot 1024 is rotatable about pin 1026, thereby allowing a practitioner of the invention to move blade 1004 along a circular direction about pin 1026. Lever 1020 is used to fix the position of blade 1004 at a desired position along that circular direction. FIG. 10D illustrates a magnified view of lever 1020, slot 1024, and blade 1004 to better illustrate the mechanism used to fix the position of blade 1004. (FIG. 10D omits other portions of retractor system 1000 for clarity). Lever 1020 includes a gradually sloping or “hour-glass” shaped portion 1030 which wedges together or contacts slot 1024 at varying levels of force depending on how far lever 1020 is rotated. In this manner, a practitioner of the invention can adjustably fix slot 1024 and blade 1004 at a desired position along a circular direction about pin 1026.

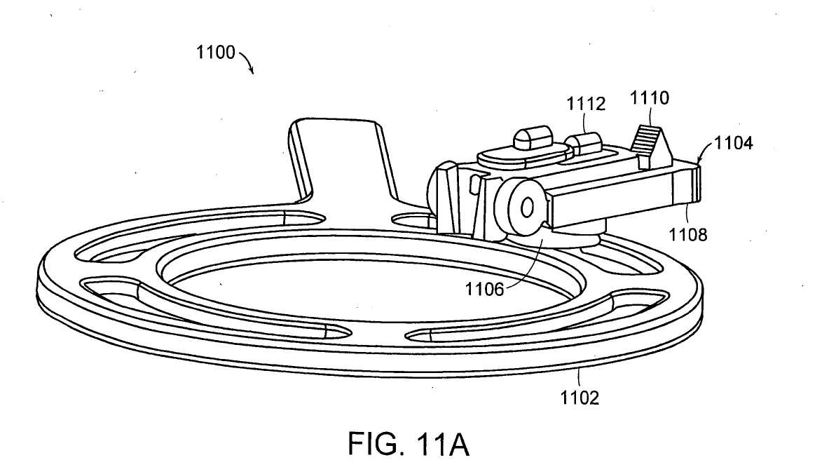

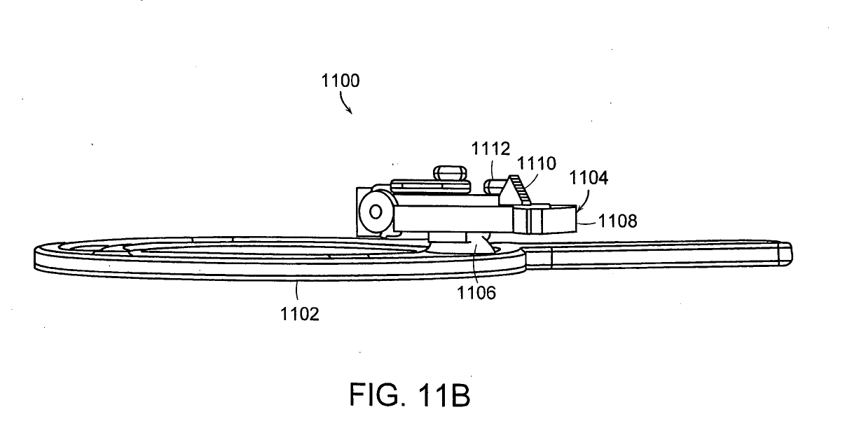

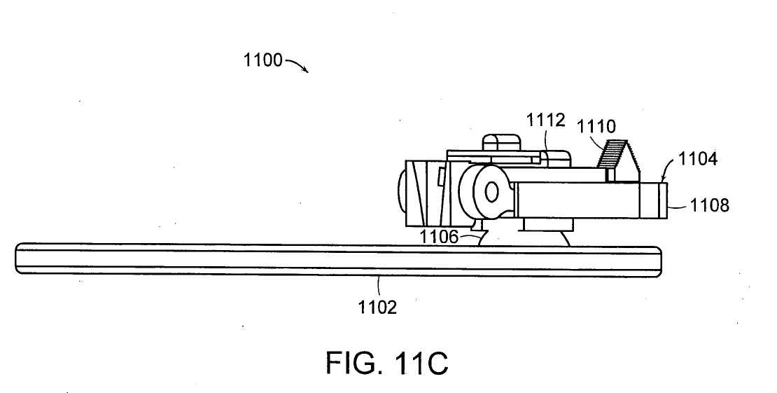

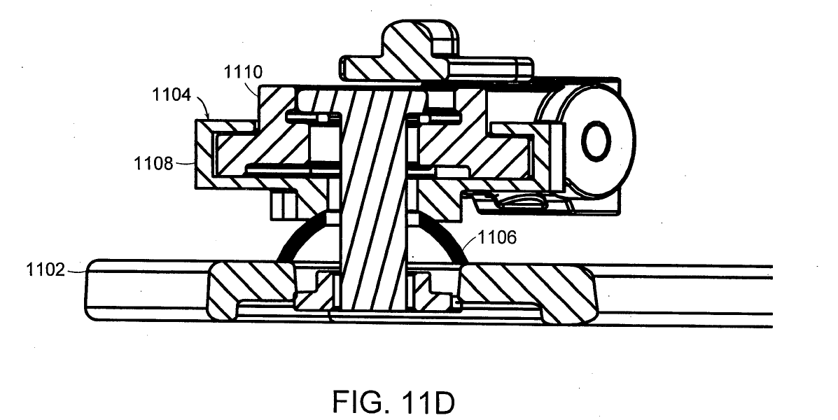

FIG. 11A illustrate another embodiment of the invention that includes retractor 1100. FIGS. 11B and 11C illustrate two side-views of retractor 1100. Retractor 1100 includes frame 1102 and a blade assembly comprising arm 1104. Arm 1104 includes polyaxial slide plate 1106. Polyaxial slide plate 1106 allows arm 1104 to be moved about the points or region of contact between arm 1104 and frame 1102 in such a way that an attached blade can be moved in a direction that circumscribes at least a portion of a sphere that is centered at the point of contact between arm 1104 and frame 1102. That is, polyaxial slide plate 1106 pivots slide base 1108, slide 1110, and any attached blade about the points of contact between arm 1104 and frame 1102, thereby allowing an attached blade to be positioned along directions that circumscribe a sphere centered on the pivot point of plate 1106. A practitioner of the invention can fix the position of the blade at a desired location on the perimeter of that sphere by rotating lever 1112. FIG. 11D illustrates a cut-away view of arm 1104 and frame 1102 of retractor 1100.

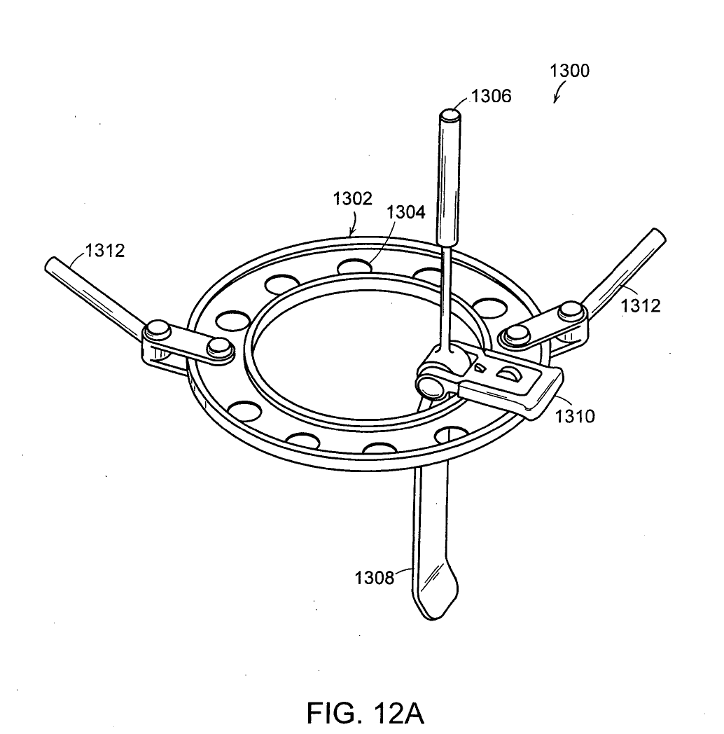

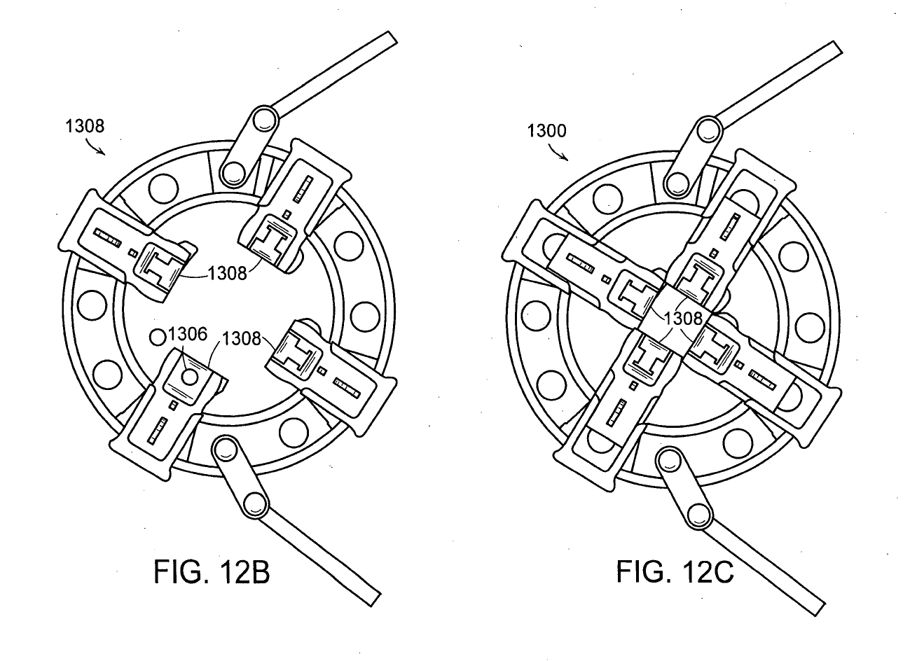

FIG. 12A illustrates a perspective view of an embodiment of the invention that includes surgical retractor 1300. Retractor 1300 includes frame 1302 which is held over a surgical site by arms 1312. Frame 1302 is circular in shape and includes a plurality of attachment points 1304. Arms 1312 can be attached to a supporting structure, which can, for example, be a table, a rack, a cart, or the like. Frame 1302 supports one or more arms 1310 that are movable along the periphery thereof. Arm 1310 adjustably supports surgical retractor blade 1308. Attachment tool 1306 can be used to attach retractor blade 1308 to arm 1310. FIG. 12B illustrates a plane view of the top of retractor 1300 with a plurality of blades 1308 in a retracted position. FIG. 12C illustrates a plane view of the top of retractor 1300 in a contracted position.

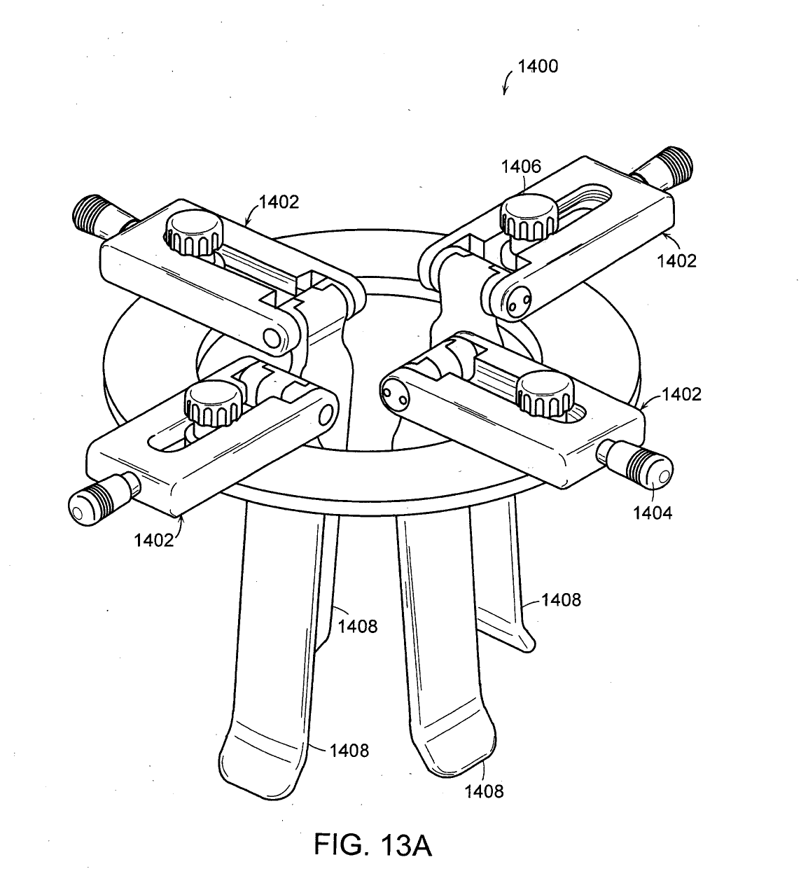

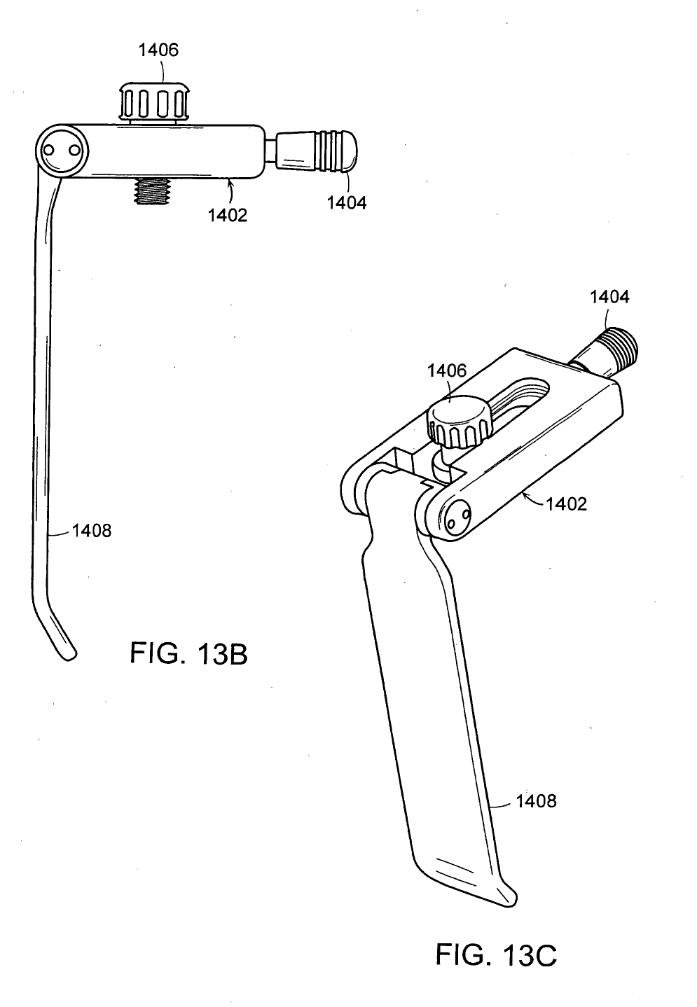

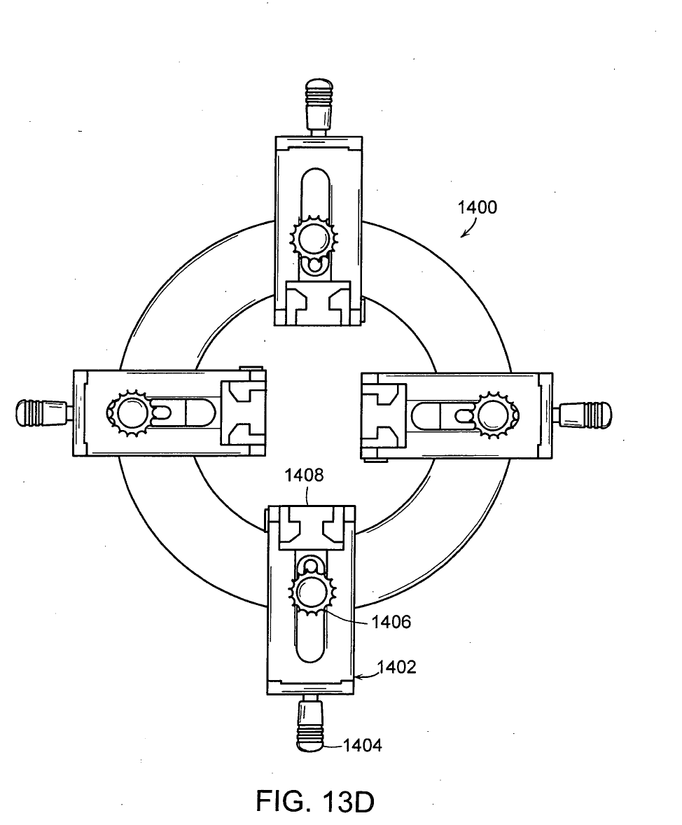

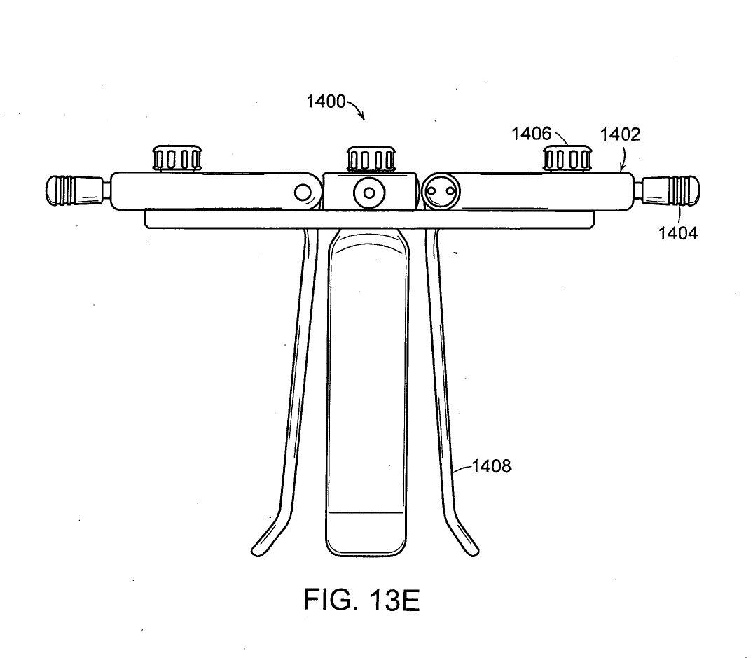

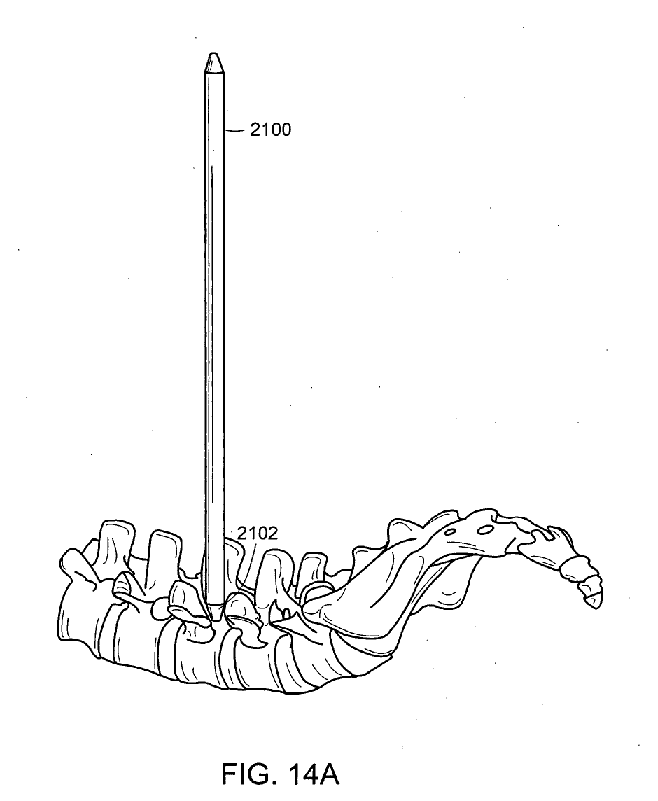

FIG. 13A illustrates another embodiment of the present invention that includes retractor 1400. Retractor 1400 includes a plurality of arms 1402 attached to a frame and blades 1408. FIGS. 13B and 13C illustrate two perspective views of a portion of retractor 1400 that includes arm 1402 attached to blade 1408. Arms 1402 includes a blade rotation locking mechanism that utilizes screws 1406 and knobs 1404 in order to fix the position of blades 1408 in at least two, non-collinear directions. Knobs 1404 can be pulled out radially from the frame to disengage teeth from grooves in a rotational blade mechanism, thereby permitting blade rotation. Springs located along the shaft of knobs 1404 apply compression on the teeth to engage the grooves within the cylinder attachment portion of blades 1408, thereby providing a continuous compression and continuously locking the rotation or toeing of blades 1408 until a practitioner disengages the teeth by pulling knobs 1404. FIG. 13D illustrate a plane view of retractor 1400, with two blades 1408 in a retracted position and two in a contracted position. FIG. 13E illustrates a side view of retractor 1400 with at least two blades 1408 in a contracted position.

The retractors of the present invention can be made of those materials that are commonly used in medical devices. Examples of suitable materials include metals and metal alloys (e.g., stainless steel, aluminum, titanium, nitinol, cobalt chrome, etc.), plastics (e.g., carbon fiber reinforced polyethylene (CFRP), ultra high molecular weight polyethylene (UHMWP), ultem, radel, vectra, polycarbonate, etc.). In some embodiments of the invention, the retractor includes a radiolucent material (e.g., radiolucent plastics, aluminum, thin stainless steel, titanium, nitinol, or cobalt chrome). In further embodiments of the invention, the retractors include radiopaque materials that can, for example, be used as markers to assist a practitioner in positioning a retractor of the invention or portions of a retractor of the invention.

In some embodiments, this invention includes methods of forming a surgical site in a mammal. The methods comprise creating an incision in the mammal, directing a retractor of the present invention through the incision and into the mammal, and retracting the tissue of the mammal at the incision with the retractor to form a surgical site. The surgical site is defined at least in part by the retractor blades. Such methods are useful for providing surgical access to portions of mammalian anatomy (e.g., a mammalian spinal column).

In some embodiments, the method includes making a first incision in the epidermis of the mammal and then expanding the incision into a portion of subdermal tissue to create a pathway in any conventional manner. For example, the first incision can be expanded by dilation to the desired size, shape, and orientation by using a plurality of dilators (e.g., at least two dilators) to create a pathway. Exemplary methods and instruments for serial dilation are described in commonly owned U.S. Pat. No. 6,159,179, entitled Cannula and Sizing and Insertion Method; U.S. patent application Ser. No. 10/024,221, filed Oct. 30, 2001, entitled Non-Cannulated Dilators; and U.S. patent application Ser. No. 10/021,809, filed Oct. 30, 2001, entitled “Configured and Sized Cannulas,” each of which are incorporated herein by reference. Once the tissue has been dilated to a desired extent, a retractor of the present invention can be inserted into the dilated incision to further expand the first incision and/or to define the pathway.

In some embodiments, the incision is expanded to create a surgical pathway using an intermuscular procedure that includes locating a muscle plane and separating the muscles at the muscle plane to create the first pathway. For example, in certain exemplary methods, the intermuscular plane separating the multifidus and longissimus muscles may be located through the first incision. The multifidus and longissimus muscles may be separated at the muscle plane by inserting a finger or an instrument, such as a retractor, through the muscle plane and advancing the finger or instrument to the vertebra to create the first pathway. Intermuscular procedures are described in detailed in U.S. Pat. No. 6,692,434, entitled Method and Device for Retractor for Microsurgical Intermuscular Lumbar Arthrodesis; U.S. patent application Ser. No. 10/060,905, filed Jan. 29, 2002, entitled Retractor and Method for Spinal Pedicle Screw Placement; and New Uses and Refinements of the Paraspinal Approach to the Lumbar Spine, L. L. Wiltse and C. W. Spencer, Spine, Vol. 13, No. 6, Nov. 6, 1988, each of which is incorporated herein by reference.

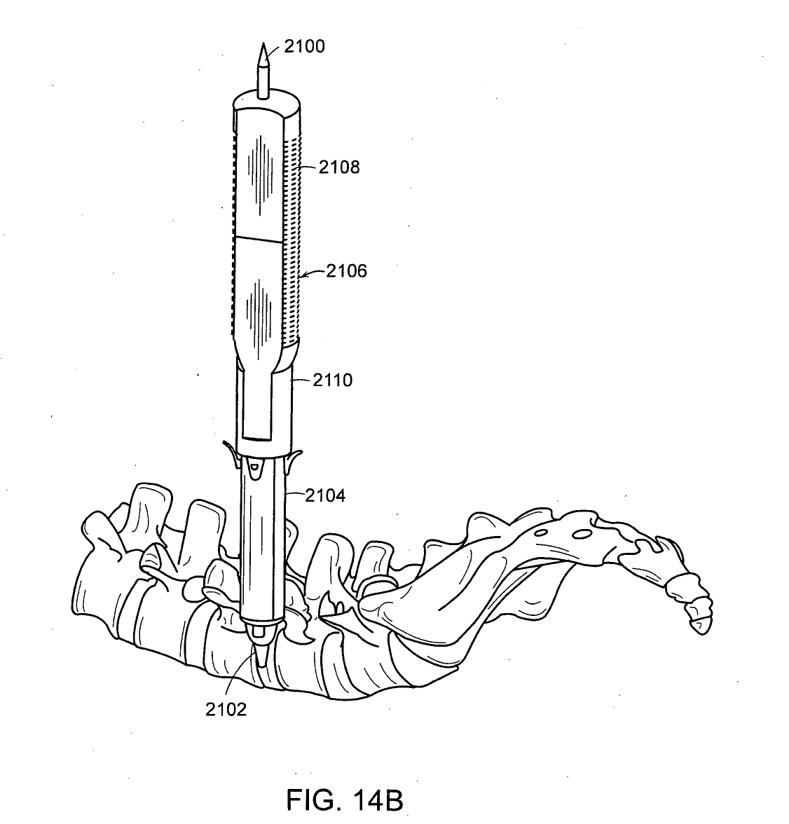

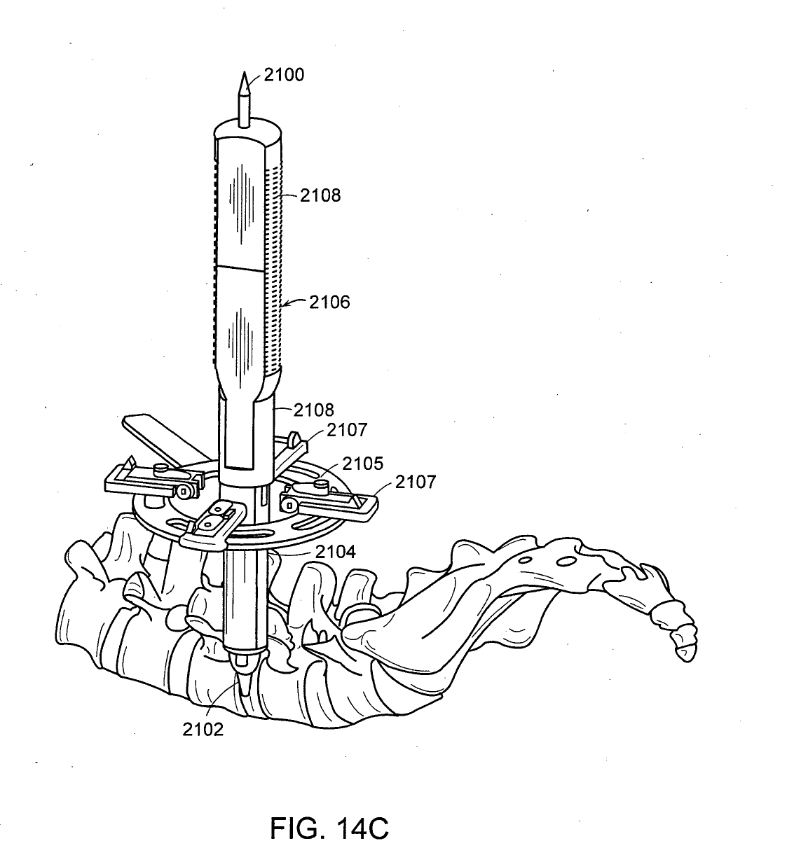

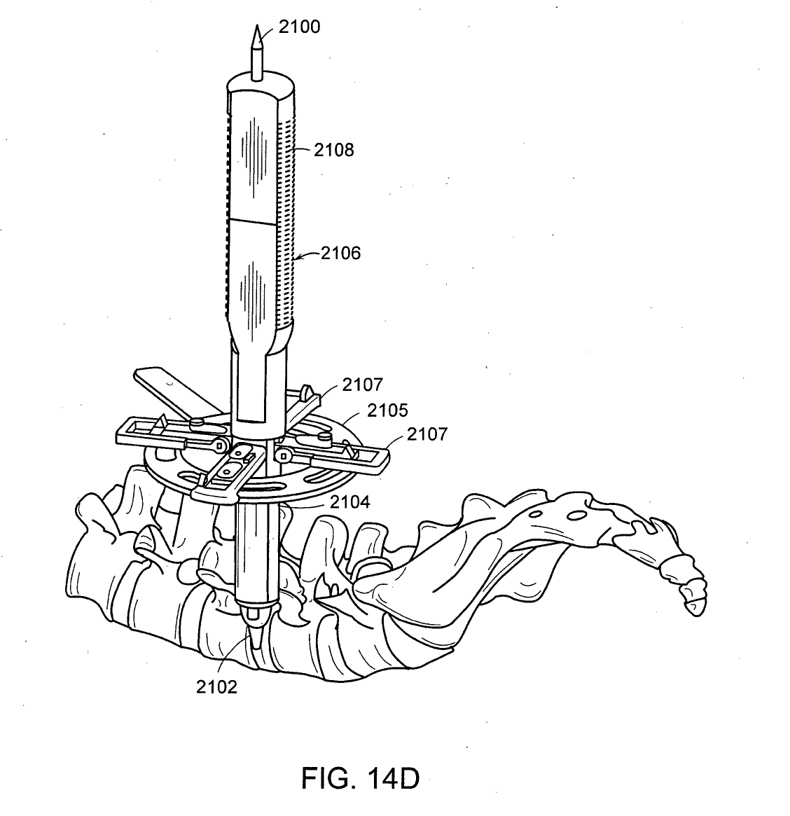

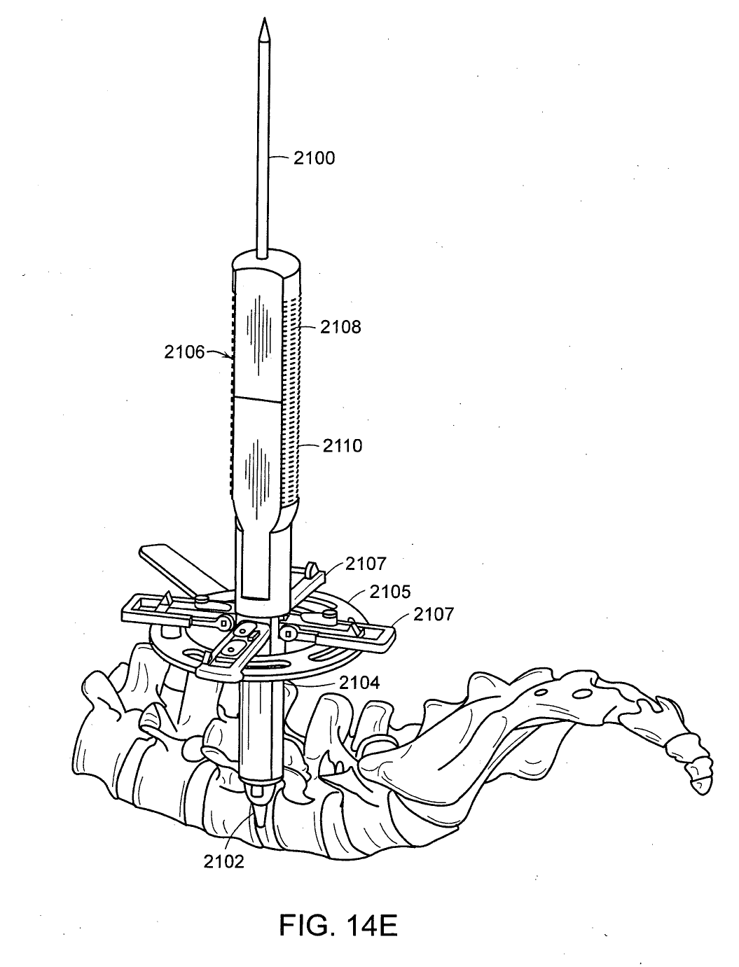

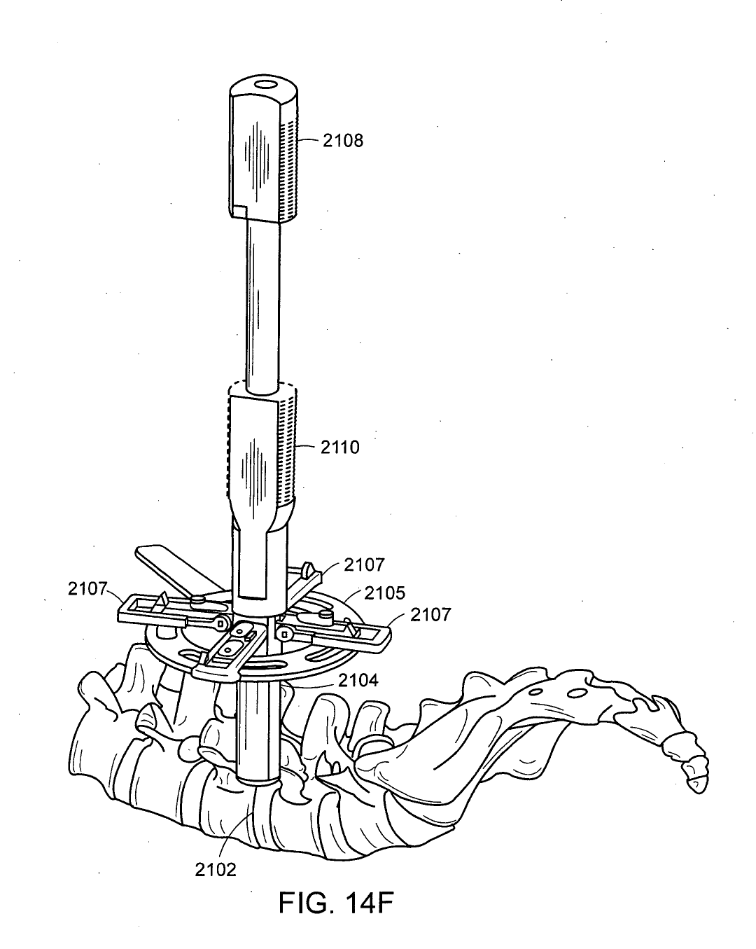

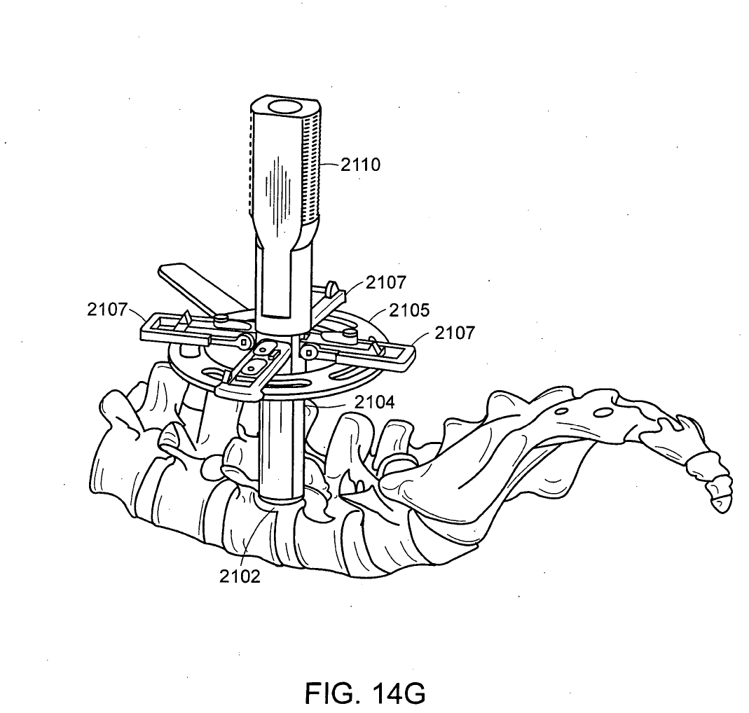

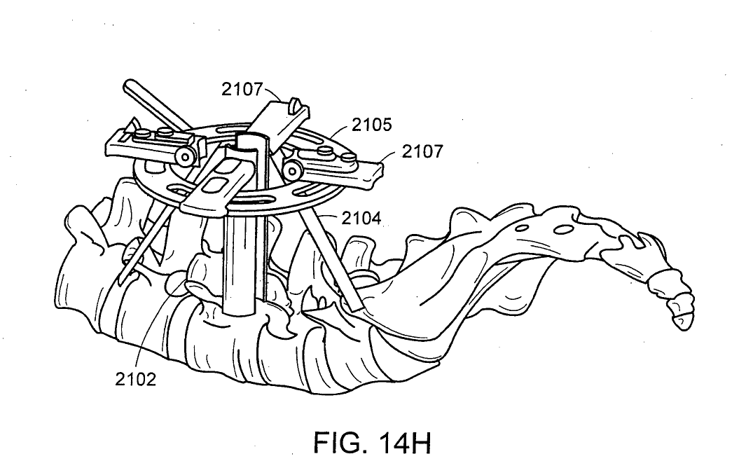

FIGS. 14A-14H illustrate one embodiment of a method of the invention where a retractor of the invention is directed into a mammal to the depth of the surgical site or near the depth of the surgical site to be formed with the use of an obtruator. Soft tissue and some bone mass has been omitted from the figures for clarity.

FIG. 14A illustrates obtruator 2100 after it has been inserted into an incision and forced down to the surgical site (i.e., near the spinal column). Optionally, the obtruator is directed along a guide wire which has previously been tethered to the surgical site.

Once obtruator 2100 is in position at surgical site 2102, retractor blades 2104 are assembled to insertion tube 2106. Insertion tube 2106 includes inner sleeve 2108 and outer sleeve 2110. Retractor blades 2104 include attachment points that complement attachment ports on outer sleeve 2110 are used to attach or assemble blades 2104 to tube 2106. The combined assembly of retractor blades 2104 and insertion tube 2106 defines a conduit that has an inner diameter that is greater than the outer diameter of obtruator 2100. This allows the combined assembly of retractor blades 2104 and insertion tube 2106 to, in turn, be assembled over obtruator 2100. Once retractor blades 2104 and tube 2106 are assembled over obtruator 2100, a surgeon or other practitioner of this embodiment pushes retractor blades 2104 down the length of obtruator 2100 to surgical site 2102 by applying force on insertion tube 2106, as shown in FIG. 14B. In some embodiments, the obtruator and insertion tube are combined into one single instrument, thereby allowing the blades to be inserted into the incision in a single step.

Once retractor blades 2104 are at surgical site 2102, retractor frame 2105 and attached arms 2107 are directed over and down tube 2106 until arms 2107 are level with proximal portions of blades 2104, as shown in FIG. 14C. Arms 2107 are then extended and blades 2104 are attached to the blade attachment points of arms 2107, as shown in FIG. 14D. In some embodiments of the invention, the entire retractor is assembled to the inserter tube prior to insertion into the incision. The entire assembly (e.g., blades, arms, frames, and inserter tube) can then be inserted into the incision in a single step. Alternatively, the entire assembly is inserted over an obtruator already in the incision.

After blades 2104 are assembled to arms 2107 and frame 2105, obtruator 2100 is removed from the incision, leaving tube 2106, retractor frame 2105, arms 2107, and retractor blades 2104 in or positioned around the incision, as shown in FIG. 14E.

Inner sleeve 2108 is then removed from insertion tube 2106, as shown in FIGS. 14F and 14G. Outer sleeve 2110 can be used to position the assembled retractor to a desired depth and position (e.g., to the surgical site or above the surgical site to allow sufficient room for subsequent positioning of one or more retractor blades).

Blades 2104 are then disassembled from outer sleeve 2110, and sleeve 2210 is removed from the incision, leaving blades 2104 at surgical site 2102 and assembled to frame 2105 and arms 2107. Blades 2104 can then be positioned as desired, as shown in FIG. 14H. Optionally, additional blades can be attached to frame 2105 and inserted or positioned in any desired order or combination (e.g., all blades inserted or positioned simultaneously or individually). When all the portions of the insertion tube 2106 have been disassembled from retractor 2104, one or more of the inserted blades 2104 can be swapped out with different or similar blades.

The surgical site is formed by the distal ends of blades 2104. Optionally, the retractor is attached to a surgical retractor positioning means (e.g. a rigid arm, not shown) which rigidly secures the retractor in the desired location.

While this invention has been particularly shown and described with references to various embodiments thereof, it will be understood by those skilled in the art that various changes in form and details may be made therein without departing from the scope of the invention encompassed by the appended claims.

What is claimed is:

1. A surgical retractor, comprising:
a frame; and
a blade assembly, the blade assembly including
an arm extending from the frame,
a blade attached to the arm and moveable relative to the frame,
and
a locking mechanism that is moveable from a first position to a second position to prevent movement of the blade in at least two, non-collinear directions.

2. The surgical retractor of claim 1, wherein at least a portion of the blade is moveable relative to the frame in at least two directions selected from the group consisting of a first direction that is parallel to a line of contact between the arm and the frame and a second direction that extends about or through a point of contact between the arm and the frame.

3. The surgical retractor of claim 2, wherein the second direction is a line that both extends through a point of contact between the arm and the frame and is collinear or parallel to a major axis of the arm.

4. The surgical retractor of claim 2, wherein the second direction is an arc.

5. The surgical retractor of claim 4, wherein the arc is coplanar or parallel with a major plane of the frame.

6. The surgical retractor of claim 2, wherein the second direction circumscribes at least a portion of a sphere that has a center at the point of contact between the arm and the frame.

7. The surgical retractor of claim 1, wherein the blade is moveable relative to the frame in a direction that extends circumferentially about a line that is normal to the major axis of the arm and parallel to the major plane of the frame.

8. The surgical retractor of claim 7, wherein the surgical retractor further includes a second locking mechanism to prevent movement of the blade in the direction that extends circumferentially about the line that is normal to the major axis of the arm and parallel to the major plane of the frame.

9. The surgical retractor of claim 1, wherein the surgical retractor further includes a polyaxial hinge at a point of contact between the arm and the frame.

10. The surgical retractor of claim 1, wherein the blade is detachable from the arm.

11. The surgical retractor of claim 10, further including a pin that attaches the blade to the arm.

12. The surgical retractor of claim 11, wherein the pin is held in place by an interference fit with the blade or the arm.

13. The surgical retractor of claim 10, wherein the blade includes a clip for removably attaching the blade to the arm.

14. The surgical retractor of claim 13, wherein the clip is a dovetail clip.

15. The surgical retractor of claim 1, wherein the blade includes a distal end and a toe-out protrusion at the distal end.

16. The surgical retractor of claim 1, wherein at least a portion of the frame is arcuate.

17. The surgical retractor of claim 16, wherein the frame is substantially circular.

18. The surgical retractor of claim 16, wherein the frame is substantially elliptical.

19. The surgical retractor of claim 1, wherein the locking mechanism is moveable from the first position to the second position to prevent movement of the blade in at least three, non-collinear directions.

20. The surgical retractor of claim 19, wherein the surgical retractor further includes a polyaxial hinge where the blade assembly is attached to the frame.

21. A method of forming a surgical site in a mammal, comprising:
creating an incision in the mammal;
directing a retractor through the incision and into the mammal, the retractor including
a frame;
a blade assembly, the blade assembly including
an arm extending from the frame, and
a blade attached to the arm and moveable relative to the frame; and
a locking mechanism that is moveable from a first position to a second position to prevent movement of the blade in at least two, non-collinear directions; and
retracting the tissue of the mammal at the incision with the retractor to form a surgical site, wherein the surgical site is defined at least in part by the blades.

22. The method of claim 21, wherein the locking mechanism is moveable from the first position to the second position to prevent movement of the blade in at least three, non-collinear directions.

23. The method of claim 21, wherein the surgical retractor further includes a polyaxial hinge where the blade assembly is attached to the frame.

24. The method of claim 21, wherein the retractor is assembled to an obtruator before the retractor is directed into the mammal.

25. The method of claim 21, wherein at least two dilators are directed into the mammal before the retractor is directed into the mammal, and the retractor is directed into the mammal along one of the dilators.

26. The method of claim 21, wherein the incision is expanded by using an intermuscular procedure.

27. The method of claim 21, wherein the surgical site provides access to the spine of a mammal.
